(19) United States
(12) Patent Application Publication
(10) Pub. No.: US 2025/0262383 A1
(43) Pub. Date: Aug. 21, 2025

(54) INJECTION SYSTEM AND METHOD

(71) Applicant: CREDENCE MEDSYSTEMS, INC., Menlo Park, CA (US)

(72) Inventors: Stephen H. Diaz, Palo Alto, CA (US); Allyson Jade Weiss, San Jose, CA (US); Alan E. Shluzas, San Carlos, CA (US)

(73) Assignee: CREDENCE MEDSYSTEMS, INC., Menlo Park, CA (US)

(21) Appl. No.: 19/202,896

(22) Filed: May 8, 2025

Related U.S. Application Data
(63) Continuation-in-part of application No. 18/377,601, filed on Oct. 6, 2023.
(60) Provisional application No. 63/645,709, filed on May 10, 2024.
(60) Provisional application No. 63/414,055, filed on Oct. 7, 2022.

(51) Int. Cl. A61M 5/315; A61M 5/32

(52) U.S. Cl. CPC A61M 5/31501; A61M 5/3293

(57) ABSTRACT

An injection system includes an injection system body, a proximal stopper member, and a distal stopper member, wherein the proximal stopper member, the distal stopper member, and the injection system body form proximal and distal drug chambers. The system further includes a plunger member configured to insert the proximal stopper member relative to the injection system body. Moreover, the system includes a needle hub assembly coupled to the syringe body at the syringe body distal end, and including a needle hub and a needle. The distal stopper member includes a ball having a lodged state and a dislodged state. When the ball is in the lodged state, the ball forms an openable barrier between the proximal and distal drug chambers. The distal stopper member is configured to selectively allow flow from the proximal drug chamber to the distal drug chamber.

The present application claims priority to U.S. Provisional Patent Application Ser. No. 63/645,709, filed on May 10, 2024 under attorney docket number CM.30045.00 and entitled “INJECTION SYSTEM AND METHOD.” The present application is also a continuation-in-part of U.S. patent application Ser. No. 18/377,601, filed on Oct. 6, 2023 under attorney docket number CM.20039.00 and entitled “INJECTION SYSTEM AND METHOD.” The present application includes subject matter similar to the subject matter described in the following co-owned U.S. patent applications: (1) U.S. Utility patent application Ser. No. 14/321,706, filed Jul. 1, 2014 and issued as U.S. Utility U.S. Pat. No. 9,814,842 on Nov. 14, 2017 under attorney docket number CM.20001.00 and entitled “SAFETY SYRINGE”; (2) U.S. Utility patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. Utility patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. Utility patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. Utility patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. Utility patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. Utility patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/798,188, filed on Feb. 21, 2020 under attorney docket number CM.20023.00 and entitled “SYSTEM AND METHOD FOR SAFETY SYRINGE”; (9) U.S. Utility patent application Ser. No. 16/435,429 filed on Jun. 7, 2019 under attorney docket number CM.20019.00 and entitled “SYSTEM AND METHOD FOR SAFETY SYRINGE”; (10) U.S. Utility patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. Provisional Patent Application Ser. No. 62/904,988 filed on Sep. 24, 2019 under attorney docket number CM.30027.00 and entitled “SYSTEM AND METHOD FOR SAFETY SYRINGE”; (13) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (14) U.S. Provisional Patent Application Ser. No. 62/682,381, filed on Jun. 8, 2018 under attorney docket number CM.30019.00 and entitled “SYSTEM AND METHOD FOR SAFETY SYRINGE”; (15) U.S. Provisional Patent Application Ser. No. 62/729,880, filed on Sep. 11, 2018 under attorney docket number CM.30021.00 and entitled “SYSTEM AND METHOD FOR SAFETY SYRINGE”; (16) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (17) U.S. Provisional Patent Application Ser. No. 63/046,517, filed on Jun. 30, 2020 under attorney docket number CM.30028.00 and entitled “SYSTEM AND METHOD FOR SAFETY SYRINGE”; (18) U.S. Provisional Patent Application Ser. No. 63/156,264, filed on Mar. 3, 2021 under attorney docket number CM.30031.00 and entitled “SYSTEM AND METHOD FOR SAFETY SYRINGE”; (19) U.S. Provisional Patent Application Ser. No. 63/193,466, filed on May 26, 2021 under attorney docket number CM.30031.00 and entitled “SYSTEM AND METHOD FOR SAFETY SYRINGE”; (20) U.S. patent application Ser. No. 18/377,601, filed on Oct. 6, 2023 under attorney docket number CM.20039.00 and entitled “INJECTION SYSTEM AND METHOD”; (21) U.S. patent application Ser. No. 18/428,979, filed on Jan. 31, 2024 under attorney docket number CM.20040.00 and entitled “INJECTION SYSTEM AND METHOD”; and (22) U.S. Provisional Patent Application Ser. No. 63/300,394, filed on Jan. 18, 2022 under attorney docket number CM.30034.00 and entitled “INJECTION SYSTEM AND METHOD”. The contents of the applications and patents identified herein are fully incorporated herein by reference as though set forth in full.

### FIELD OF THE DISCLOSURE

The present disclosure relates generally to injection systems, devices, and processes for facilitating various levels of control over fluid infusion, and more particularly to systems and methods related to multiple chamber injection systems, with or without safety features, in healthcare environments.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a locked position relative to the syringe body (4). Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer and/or a combination thereof. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

In some cases, multi-component injection systems may mix injectable components (e.g., liquids and/or powders) before injection. Some systems utilize a single injection device to draw a component liquid from one container and inject the liquid component into another container to solubilize the dry component therein. The solubilized dry component is then drawn into the injection device for injection into a patient. Such systems require much handling of unsheathed needles, leading to unnecessary exposure of a user to one or more uncapped needles. Further, manually transferring the liquid component from one container to another can result in incomplete transfer of the liquid component and affect the ratio of the components in the final mixed injectable. Moreover, accessing and manipulating multiple containers of components complicates the injection process, thereby increasing the risk of user error. Accordingly, there exists a need for multi-component injection systems that simplify the manual accessing and mixing of multiple components from multiple containers.

These limitations are addressed by multiple chamber injection systems configured to mix and inject multiple components as disclosed in U.S. patents application Ser. No. 14/696,342, Ser. No. 15/801,259, and 63/300,394, which were previously incorporated by reference herein. However, there remains a need for precise control of multiple chamber injection systems for accurate handling, mixing, and delivery of multi-component injectables. For instance, there remains a need for increased and predictable fluid flow around an outer surface of a needle that has pierced through an elastic stopper member to fluidly couple two fluid chambers on opposite sides of the elastic stopper member.

In addition, an increasing number of injectable liquids (e.g., medicines) have yet another requirement that time of exposure of the injectable liquid to metals (e.g., stainless steel of a needle) be minimized.

It is also desirable to incorporate needle stick prevention technology into the injection system. The ability to retract the sharp end of the needle at least partially inside of the syringe protects the person giving the injection and the patient from inadvertent needle stick injuries.

There is a need for injection systems which address the shortcomings of currently-available configurations. In particular, there is a need for multiple chamber safety injection solutions with precise control, which may utilize the existing and relatively well-controlled supply chain of conventionally delivered prefilled syringe assemblies such as those described in reference to FIGS. 5A and 5B.

### SUMMARY

Embodiments are directed to injection systems. In particular, the embodiments are directed to multiple chamber safe injection systems with precise control of handling, mixing, and delivery of multi-component injectables.

In one embodiment, an injection system includes an injection system body defining a proximal opening at a proximal end thereof and a distal needle interface at a distal end thereof. The system also includes a proximal stopper member and a distal stopper member disposed in the injection system body, wherein the proximal stopper member, the distal stopper member, and the injection system body form a proximal drug chamber between the proximal stopper member and the distal stopper member, and a distal drug chamber between the distal stopper member and the distal end of the injection system body. The system further includes a plunger member configured to insert the proximal stopper member relative to the injection system body. Moreover, the system includes a needle hub assembly coupled to the syringe body at the syringe body distal end, and including a needle hub, and a needle removably coupled to the needle hub and having a sharp needle distal end and a needle proximal end feature. The distal stopper member includes a ball having a lodged state and a dislodged state. When the ball is in the lodged state, the ball forms an openable barrier between the proximal and distal drug chambers. The distal stopper member is configured to selectively allow flow from the proximal drug chamber to the distal drug chamber.

In one or more embodiments, the distal stopper member defines a ball trap disposed proximal of the ball when the ball is in the lodged state. The distal stopper member may include a plurality of ball trap flaps separated by ball trap slots. The ball trap flaps may be configured to retain the ball in the ball trap when the ball is in the dislodged state.

In one or more embodiments, the distal stopper member defines a longitudinal space configured to hold the ball in a proximal end thereof when the ball is in the lodged state. The distal stopper member may include a plurality of inwardly extending exit ribs at the proximal end of the longitudinal space. The plurality of inwardly extending exit ribs may be configured to hold the ball in the proximal end of the longitudinal space when the ball is in the lodged state.

In one or more embodiments, the distal stopper member defines a plurality of flow channels along an interior wall of a distal end of the longitudinal space. The distal stopper member may include a plurality of inwardly extending centering ribs between corresponding pairs of flow channels. The plurality of inwardly extending centering ribs may be configured to align the needle in the longitudinal space. The plurality of inwardly extending centering ribs may be configured to retain the ball in the proximal end of the longitudinal space when the ball is in the lodged state. Each of the plurality of inwardly extending centering ribs may define a respective rib recess configured to retain the ball in the proximal end of the longitudinal space when the ball is in the lodged state. The distal stopper member may define a distally opening funnel at a distal end thereof. The distally opening funnel may be configured to guide the needle proximal end feature and the needle into a distal opening of the longitudinal space. While the distal stopper member in some embodiments include a plurality of inwardly extending centering ribs, in other embodiments the distal stopper does not include centering ribs.

In one or more embodiments, the plunger member includes a plunger member body defining a plunger interior. The plunger member may include a needle retention member disposed in the plunger interior and configured to couple to the needle proximal end feature. The plunger member may include an energy-storage member disposed in the plunger interior and configured to withdraw the needle retention member proximally in the plunger interior. The plunger member may include an energy-storage member latching member disposed in the plunger interior and having a latched state in which the energy-storage member latching member holds the energy-storage member in an energized state, and an unlatched state in which the energy-storage member latching member does not restrain the energy-storage member and the energy-storage member transforms into a released state. When the distal stopper member is inserted to the syringe body distal end, the energy-storage member latching member is transformed from the latched state to the unlatched state, and the energy-storage member pulls needle proximally such that the sharp needle distal end is disposed in an interior of the injection system body. When the distal stopper member is inserted to the syringe body distal end, the energy-storage member latching member may be transformed from the latched state to the unlatched state, and the energy-storage member may pull the needle proximally via the needle retention feature coupled to the needle proximal end feature.

In one or more embodiments, the distal stopper member includes a distally opening funnel insert at a distal end thereof. The distally opening funnel insert is configured to guide the needle proximal end feature and the needle into a distal opening of the longitudinal space. The distally opening funnel insert may define a central opening sized and shaped to allow the needle proximal end feature to pass therethrough. The distally opening funnel insert may include a plurality of ribs configured to support the distally opening funnel insert in the distal stopper member. The distally opening funnel insert and the distal stopper member may define a plurality of arcuate slots adjacent an outer circumference of the distally opening funnel insert configured to provide low liquid flow resistance through the distal stopper member. The distally opening funnel insert may be formed from a polymer material such as cyclic olefin polymer (COP) or cyclic olefin copolymer (COC). In some embodiments, the distally opening funnel may be formed from other polymer materials, metal, ceramic, or glass, materials, or a combination thereof.

In another embodiment, a method for injecting includes providing a prefilled injection system. The injection system includes an injection system body defining a proximal opening at a proximal end thereof and a distal needle interface at a distal end thereof. The injection system also includes a proximal stopper member and a distal stopper member disposed in the injection system body, wherein the proximal stopper member, the distal stopper member, and the injection system body form a proximal drug chamber between the proximal stopper member and the distal stopper member, and a distal drug chamber between the distal stopper member and the distal end of the injection system body. The injection system further includes a plunger member configured to insert the proximal stopper member relative to the injection system body. Moreover, the injection system includes a needle hub assembly coupled to the syringe body at the syringe body distal end, and including a needle hub, and a needle removably coupled to the needle hub and having a sharp needle distal end and a needle proximal end feature. In addition, the injection system includes a first drug component disposed in the proximal drug chamber, and a second drug component disposed in the distal drug chamber. The distal stopper member includes a ball having a lodged state and a dislodged state. The method also includes moving the plunger and the proximal stopper member coupled thereto distally into the injection system body to move the distal stopper member distally into the injection system body and to dispose the needle proximal end feature in the distal stopper member adjacent the ball. The method further includes moving the plunger and the proximal stopper member coupled thereto distally further into the injection system body to dislodge the ball from the distal stopper member using the needle proximal end feature. Moreover, the method includes moving the plunger and the proximal stopper member coupled thereto distally yet further into the injection system body to transfer the first drug component from the proximal drug chamber to the distal drug chamber to form a mixed drug with the second drug component. In addition, the method includes moving the plunger and the proximal stopper member coupled thereto distally still further into the injection system body to eject the mixed drug from the distal drug chamber through the needle.

In one or more embodiments, the method includes automatically retracting the needle proximally after ejecting the mixed drug from the distal drug chamber such that the sharp needle distal end is disposed in an interior of the injection system body.

The aforementioned and other embodiments of the invention are described in the Detailed Description which follows.

### BRIEF DESCRIPTION OF THE DRAWINGS

FIGS. 1A to 5C illustrate various aspects of conventional injection syringe configurations.

FIGS. 6 and 6A are perspective and detailed perspective views illustrating various aspects of syringe based dual chamber safe injection systems according to some embodiments.

FIGS. 7A to 7F are side views illustrating various aspects of safe syringe based dual chamber safe injection systems during steps in methods for mixing and injecting using same according to some embodiments.

FIGS. 8 to 20 are longitudinal cross-sectional and detailed longitudinal cross-sectional views illustrating various aspects of safe syringe based dual chamber safe injection systems during steps in methods for mixing and injecting using same according to some embodiments.

FIG. 21 is a detailed longitudinal cross-sectional view illustrating various aspects of stopper members with a ball disposed therein for use with safe syringe based dual chamber injection systems according to some embodiments.

FIG. 22 is a detailed longitudinal cross-sectional view illustrating various aspects of stopper members for use with safe syringe based dual chamber injection systems according to some embodiments.

FIGS. 23A and 23B are detailed distal and proximal axial views illustrating various aspects of stopper members for use with safe syringe based dual chamber injection systems according to some embodiments.

FIGS. 24A and 24B are a detailed distal perspective cross-sectional view and a detailed distal axial view illustrating various aspects of stopper members for use with safe syringe based dual chamber injection systems according to some embodiments.

FIG. 25 is a distal perspective view illustrating various aspects of syringe based dual chamber safe injection systems according to some embodiments.

FIG. 26 is a detailed distal perspective cross-sectional view illustrating various aspects of syringe based dual chamber safe injection systems according to some embodiments.

FIG. 27 is a distal perspective view illustrating various aspects of syringe based dual chamber safe injection systems according to some embodiments.

FIG. 28 is a proximal perspective view illustrating various aspects of stopper members for use with safe syringe based dual chamber injection systems according to some embodiments.

FIG. 29 is a proximal axial view illustrating various aspects of stopper members for use with safe syringe based dual chamber injection systems according to some embodiments.

FIG. 30 is a distal perspective view illustrating various aspects of stopper members for use with safe syringe based dual chamber injection systems according to some embodiments, with various components omitted for clarity.

FIG. 31 is a side view illustrating various aspects of a needle for use with safe syringe based dual chamber injection systems according to some embodiments.

FIG. 32 is a side view illustrating various aspects of a needle and a stopper member insert for use with safe syringe based dual chamber injection systems according to some embodiments.

FIGS. 33 and 33A are distal and detailed distal perspective views illustrating various aspects of safe syringe based dual chamber injection systems and stopper members for use with same according to some embodiments.

FIG. 34 is a distal axial view illustrating various aspects of stopper members for use with safe syringe based dual chamber injection systems according to some embodiments.

FIG. 35 is a detailed perspective cross-sectional view illustrating various aspects of safe syringe based dual chamber injection systems and stopper members for use with same according to some embodiments.

FIG. 36 is a distal perspective view illustrating various aspects of funnel inserts for use with stopper members in safe syringe based dual chamber injection systems according to some embodiments.

FIGS. 37 and 38 are proximal perspective views illustrating various aspects of funnel inserts for use with stopper members in safe syringe based dual chamber injection systems according to some embodiments.

FIG. 39 is a distal axial view illustrating various aspects of stopper members for use with safe syringe based dual chamber injection systems according to some embodiments.

FIGS. 40 and 41 are detailed perspective and detailed perspective cross-sectional views illustrating various aspects of stopper members having ball valves for use in safe syringe based dual chamber injection systems according to some embodiments.

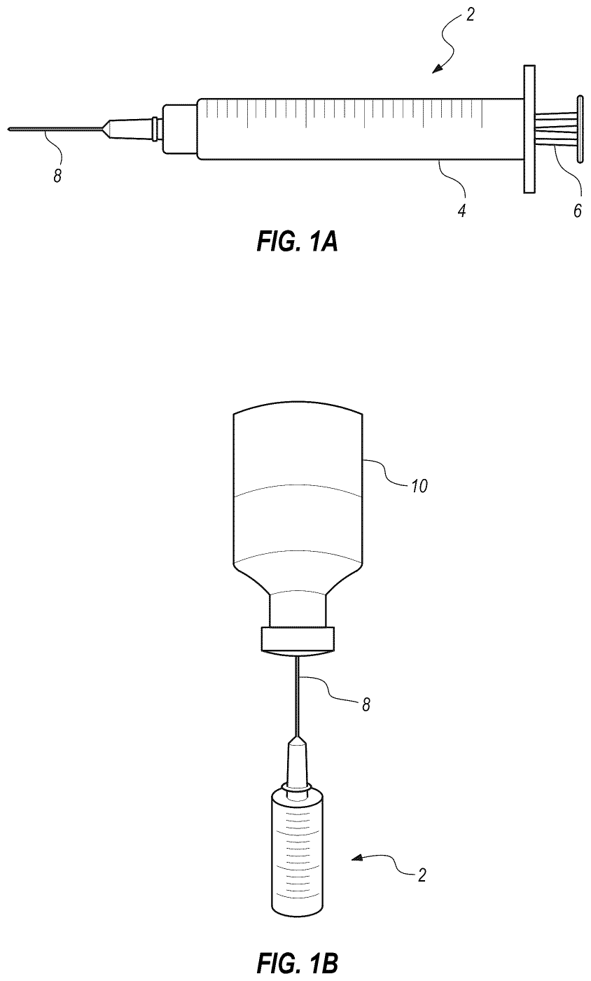

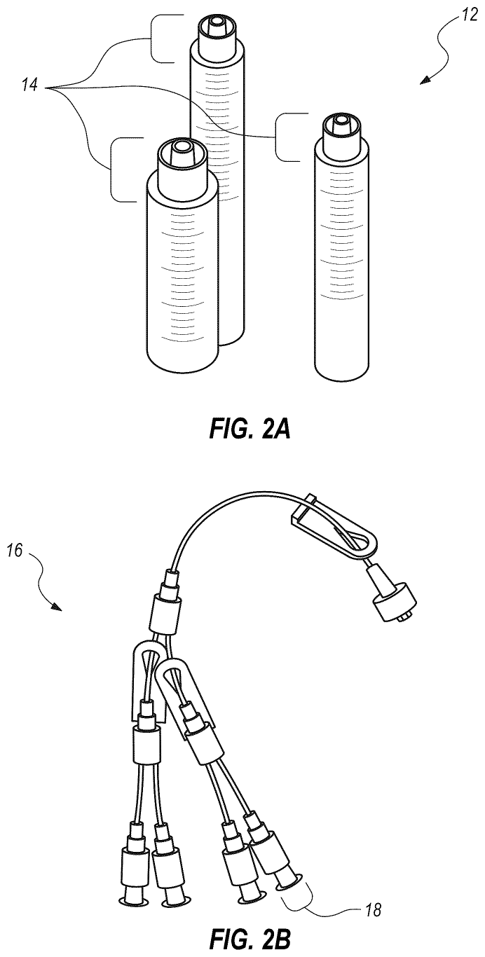

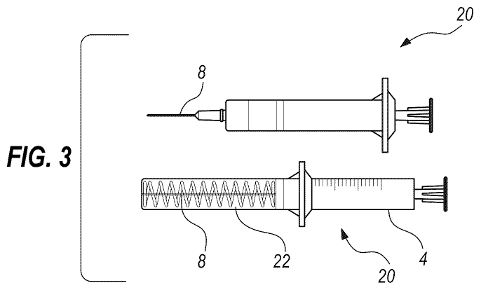

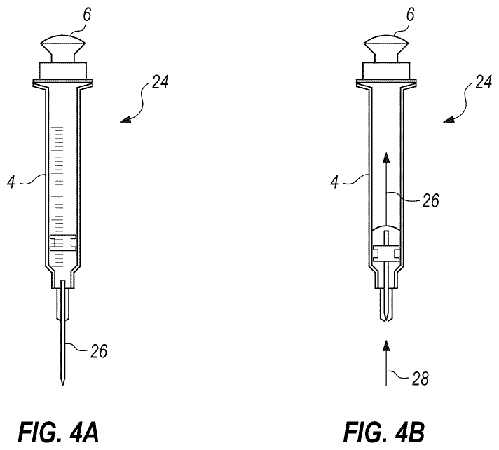

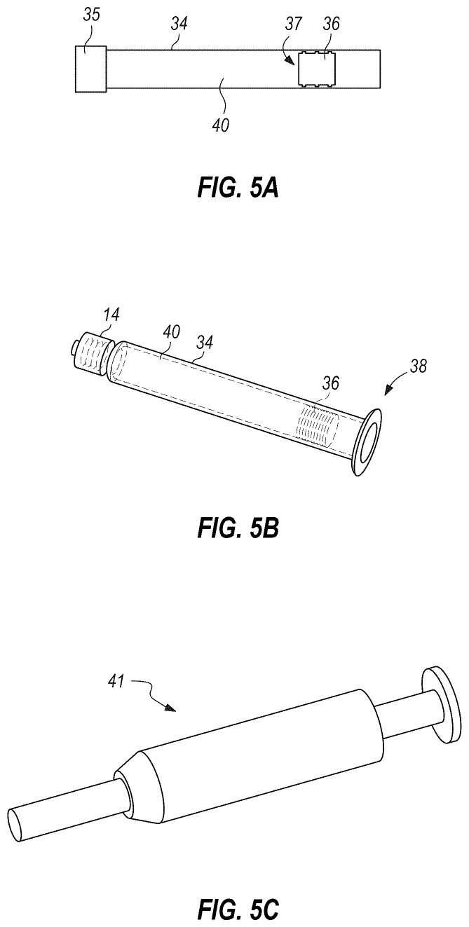

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Prefilled Dual Chamber Safe Injection Systems

### Exemplary Dual Chamber Safe Syringe Systems

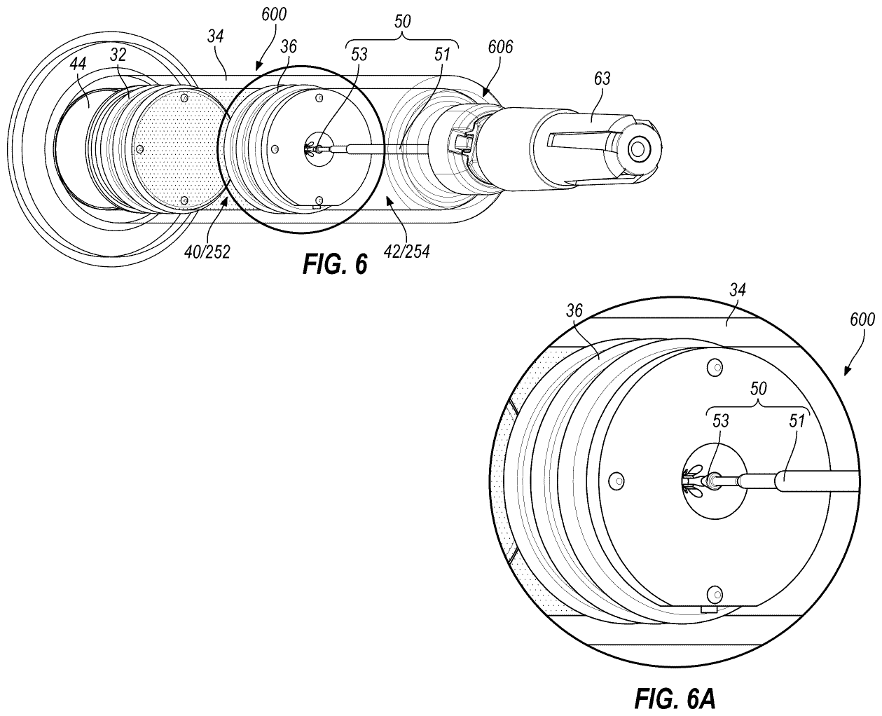

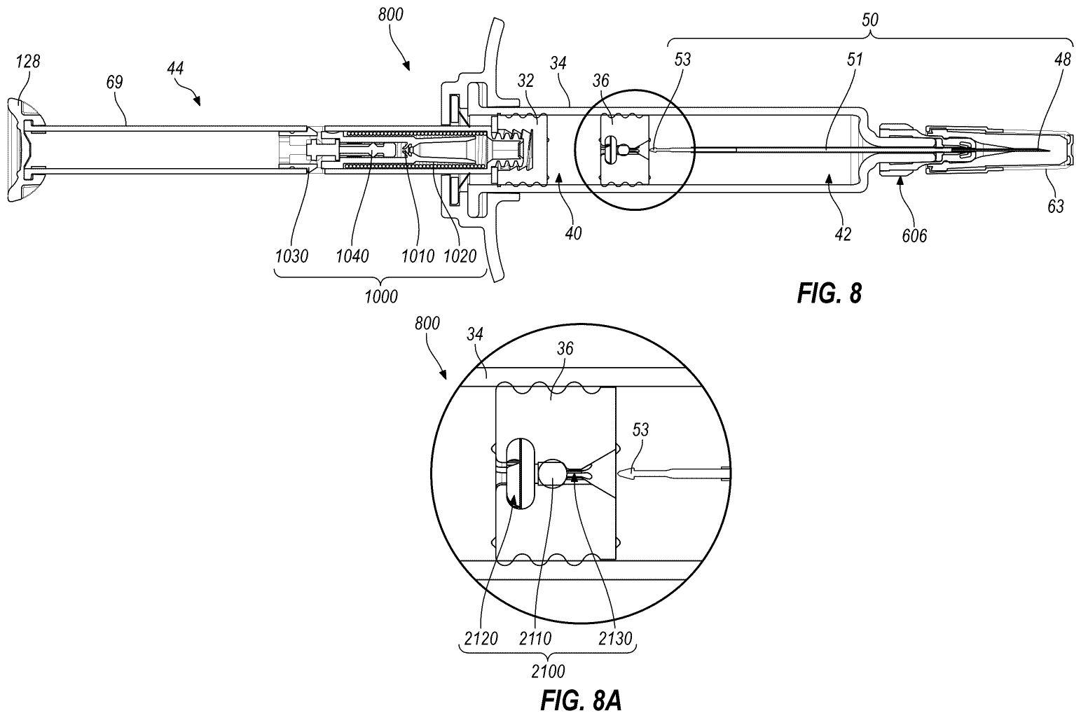

Referring to FIGS. 6 and 6A, a perspective and a detailed perspective view of a dual chamber safe injection system (600) are shown, with a conventional off-the-shelf pre-filled syringe body (34) with a conventional proximal stopper member (32) and a distal stopper member (36) disposed therein. The proximal and distal stopper members (32, 36) together with the syringe body (34) define proximal and distal drug chambers (40, 42). The proximal and distal stopper members (32, 36) define the proximal and distal ends of the proximal drug chamber (40). The distal stopper member (36) defines a proximal end of the distal drug chamber (42). A needle hub assembly (606) is disposed at the distal end of the distal drug chamber (42) with a needle cover member (63) installed for storage. The dual chamber safe injection system (600) controls transfer of a first drug component from the proximal drug chamber (40) to the distal drug chamber (42) and exit of a combined/mixed drug from the distal drug chamber (42) distally subject to sequential insertion of a plunger member (44) relative to the syringe body (34) to various degrees by a user. The plunger member (44) includes the proximal stopper member (32), a plunger housing member (69) and a plunger manipulation interface (128) (see e.g., FIG. 8). The first drug component (252) located in the proximal drug chamber (40) may be a liquid such as aqueous or oil based medicine solutions, a gel, or the first drug component may be a diluent for mixing with the second drug component (254) in the distal drug chamber (42). The second drug component (254) in the distal drug chamber (42) may be a dry form medicine such as a powder, microspheres, emulsion, lyophilized or freeze dried medicine, or a cake like solid medicine.

In some embodiments, the plunger member (44) may be configured to be manually manipulated to insert the proximal stopper member (32) relative to the syringe body (34). In some embodiments, the plunger member (44) may be configured to be inserted using a spring or a motor of an injection device such as an auto injector. In some embodiments, the plunger member (44) may be configured to be inserted using a pen injection system.

The dual chamber safe injection system (600) has a staked needle configuration wherein upon presentation to the user, a needle hub assembly (606), comprising a needle hub and a needle member (50), including a sharp needle distal end/tip (48, see e.g., FIG. 8), a needle joining member (51) and a needle proximal end (53), are mounted in position ready for injection after removal of a needle cover member (63) which may comprise an elastomeric sealing material on its internal surface to interface with the sharp needle distal end (48) or the needle hub during storage. Alternatively, the needle cover member (63) may comprise a vent (not shown) for allowing pressure resulting from the transfer and mixing of the drug components to escape from inside the syringe body (34) while preventing contamination from entering the syringe body (34). While, the staked needle is depicted as mounted in position, the needle may be removably coupled to the syringe body (34) using a Luer interface (not shown), with the needle proximal end (53) of the needle member (50) extending through the Luer interface and into the distal drug chamber (42). Alternatively, in other embodiments, the needle may be fixedly or removably mounted to the flange on a cartridge body instead of a syringe. Such cartridge injection systems are disclosed in U.S. Utility patent application Ser. No. 15/801,281, which was previously incorporated by reference herein. In the embodiments depicted in FIGS. 6-20, a significant portion of the safe needle retraction hardware resides within a plunger housing (69).

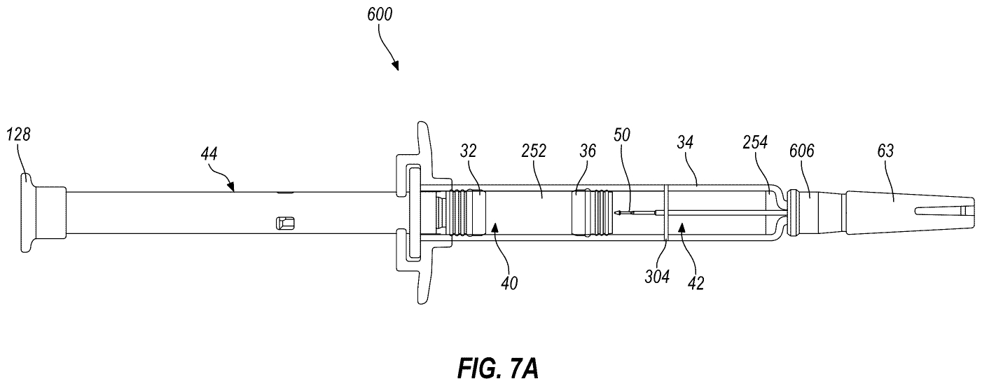

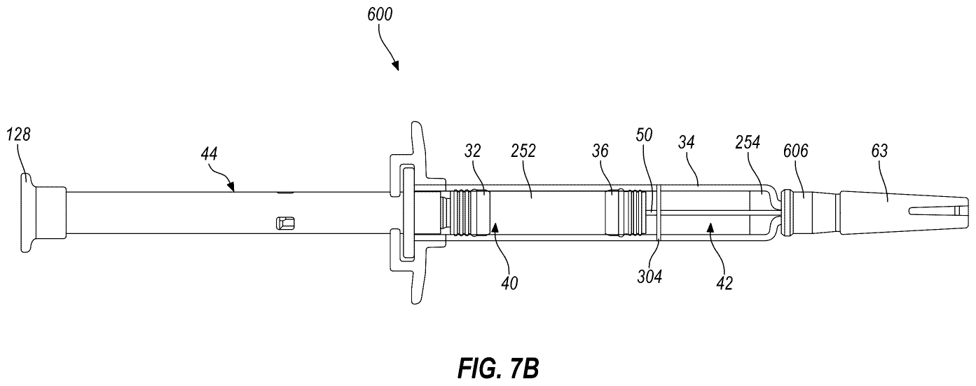

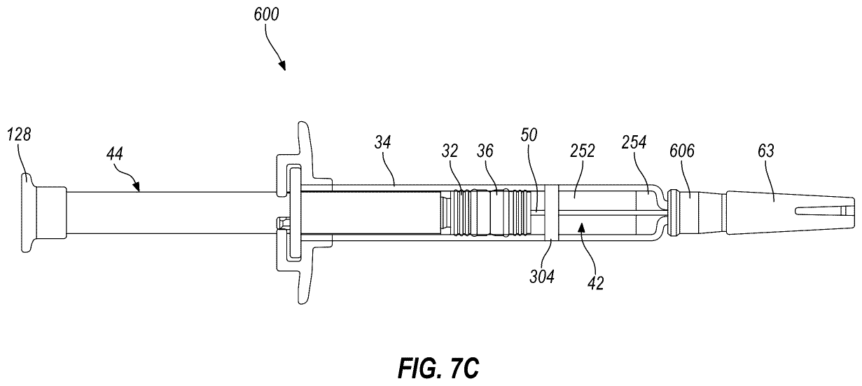

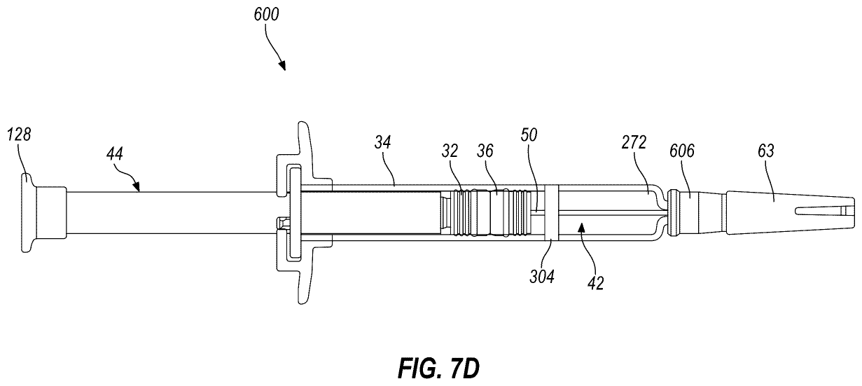

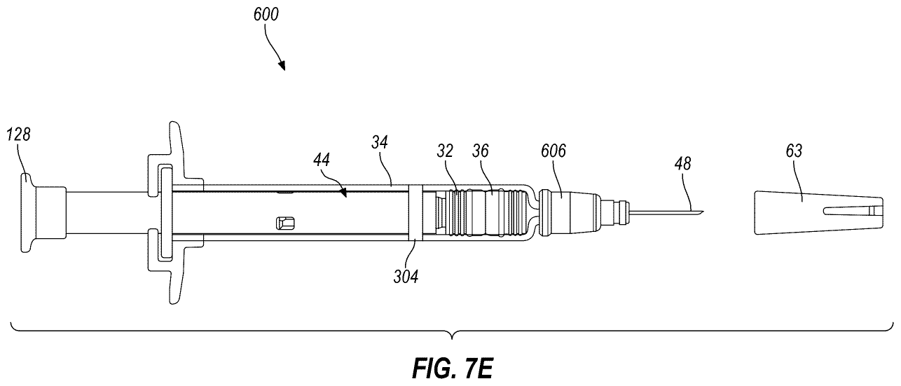

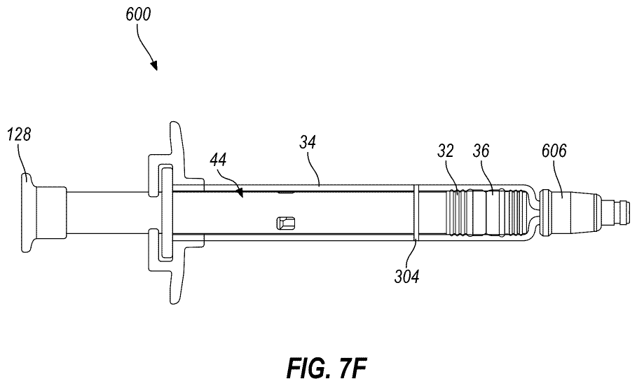

Referring to FIGS. 7A-7F, various aspects of configurations designed to facilitate injection of multi-part medications and retractions of a needle into a syringe body (34) are illustrated, wherein two or more medication components are combined to form an injection combination or solution shortly before delivery into the patient. In one variation, a liquid first drug component/diluent (252) may be combined with a substantially non-liquid second drug component (254), such as a powdered form, of a drug agent, such as a freeze-dried or lyophilized drug component, shortly before injection. The configurations described herein in reference to FIGS. 7A-7F relate to dual-chamber configurations, wherein two or more chambers within the same syringe body (34) are utilized to carry, mix, and inject an injection solution.

FIGS. 7A-7F illustrate a sequence of actions for an injection procedure utilizing a dual chamber safe injection system (600) such as that described herein. Referring to FIG. 7A, a dual chamber safe injection system (600) is in a stable configuration wherein it may be shipped or brought to an injection patient care scenario; a first drug component/liquid diluent (252) is isolated from a second non-liquid drug component (254), both within a syringe body (34) on opposite sides of a distal stopper member (36).

Referring to FIG. 7A, proximal and distal drug chambers (40, 42) are formed by a distal stopper member (36) in between two portions of the interior of a syringe body (34), such that the distal drug chamber (42) contains an air or gas gap, as well as a non-liquid medication (254); a proximal drug chamber (40), on the opposite side of the distal stopper member (36) contains a liquid diluent (252), which is proximally contained by a proximal stopper member (32). The liquid diluent (252) is a first component of a medicine and the non-liquid medication (254) is a second component of the medicine. While the distal drug chamber (42) is described as containing a non-liquid medicine (254) as a preferred embodiment, the distal drug chamber (42) may contain a liquid, solid, or gel medicine intended to be mixed with the liquid diluent (252) in the proximal drug chamber (40).

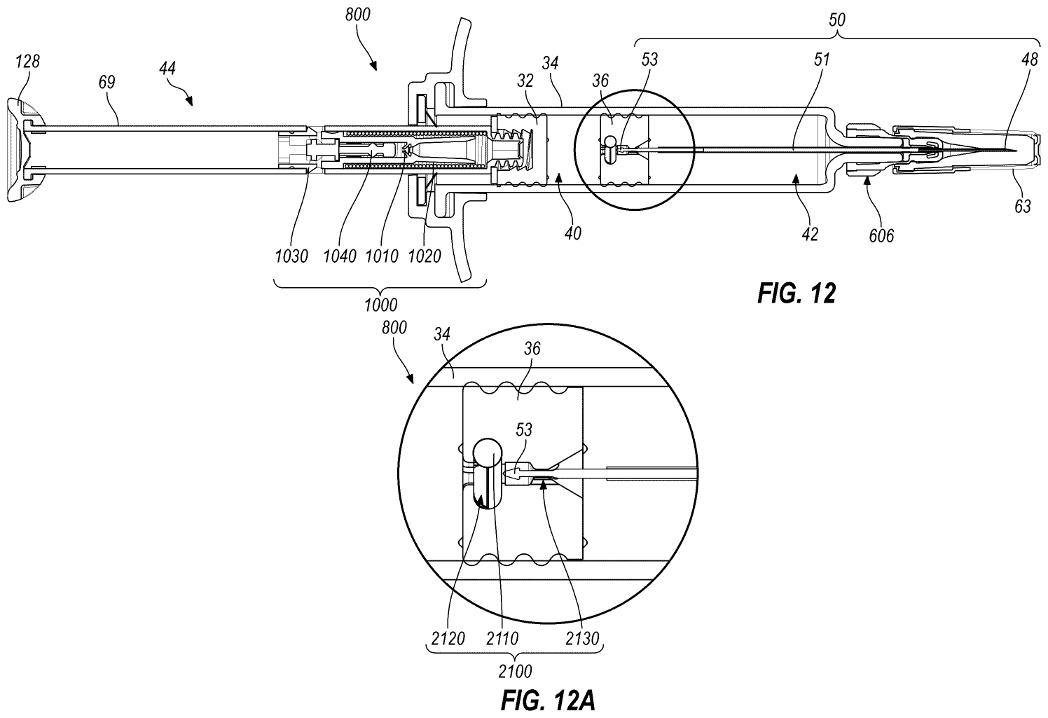

FIG. 7B illustrates initial insertion movement of the plunger member (44), advancing the distal (36) and proximal (32) stopper members together relative to the syringe body (34). With advancement sufficient to stab the needle proximal end (53) of the needle assembly across the distal stopper member (36), a fluid pathway is formed between the two previously isolated chambers (40, 42) of the syringe body (34) (see FIGS. 12 and 12A, described below).

FIG. 7C illustrates that with further insertion of the plunger member (44) until the stopper members (36, 32) are immediately adjacent each other, the liquid first drug component/diluent (252) has moved into the distal drug chamber (42) to join the non-liquid second drug component (254). FIG. 7D illustrates that with time and/or manual agitation, the liquid first drug component/diluent (252) and previously non-liquid second drug component (254) become mixed to form a mixed medication solution (272). The syringe body (34) includes a visual mixed configuration indicator (304). The mixed configuration indicator (304) is a ring disposed (e.g., painted, etched, etc.) on the syringe body (34) that indicate the approximate optimal location of the distal edge of the distal stopper member (36) when the system is in a mixed configuration in which the mixed medication solution (272) has been formed (compare FIG. 7A to FIG. 7D).

In some embodiments, especially with lyophilized non-liquid second drug components, the mixed medication solution (272) may be formed with minimal or no agitation or time passage. In another embodiment, especially with drugs which are held in suspension or emulsified drugs, vigorous shaking may be necessary to facilitate mixing. In the case of vigorous shaking it is useful to the user to be able to remove their thumb from the plunger manipulation interface (128). During transfer of liquid first drug component (252) from the proximal to the distal drug chambers (40, 42) pressure may build up in the distal drug chamber (42). This pressure acts upon the proximal and distal stopper members (32, 36) to resist stopper motion. The pressure buildup may also move the stopper members (32, 36) and plunger manipulation interface (128) proximally if the user does not have their thumb restraining the plunger member (44). Mixed configuration latches or “mix clicks” in the plunger member (44) (described in U.S. Utility patent application Ser. No. 15/801,259, which was previously incorporated by reference herein) may be utilized to provide resistance to plunger manipulation interface (128) motion due to pressure buildup and allow the user to release their thumb from the plunger manipulation interface (128) for shaking or mixing of the drug. The mix clicks may also provide an audible and/or tactile indication that the transfer of liquid first drug component (252) has been completed. The distal drug chamber (42) may also include an agitation device (not shown), which assists in mixing of the drug components.

With the assembly ready for injection of the mixed solution (272), the needle cover member (63) may be removed and the patient may be injected with the exposed needle distal end (48) with depression/insertion of the plunger member (44) and associated stopper members (36, 32) as shown in FIG. 7E. Referring to FIG. 7F, with full depression/insertion of the plunger member (44) and associated stopper members (32, 36), the sharp needle distal end/point (48, see e.g., FIG. 7E) may automatically retract to a safe position within the syringe body (34), the needle hub assembly (606), or the plunger member (44). Automatic retraction of the needle at least partially within the plunger is described in U.S. utility patent application Ser. No. 14/696,342, which was previously incorporated by reference herein.

Further details regarding multiple chamber injection systems (components, methods using same, etc.) are disclosed in U.S. Utility patent application Ser. No. 15/801,259, and U.S. Provisional Patent Application Ser. Nos. 62/682,381 and 62/729,880, which were all previously incorporated by reference herein.

### Exemplary Safe Dual Chamber Injection Systems With Stopper Members Including Ball Valves

FIGS. 8 and 8A depict a safe dual chamber injection system (800) having a ball valve (2100) disposed therein according to some embodiments. FIGS. 8 and 8A depict the injection system (800) in a storage/transport configuration. While the injection system (800) is depicted without dual chamber components for clarity, the ball valve (2100) is configured for use with any multiple chamber injection systems. The injection system (800) as shown includes an injection system body (34), a proximal stopper member (32), a distal stopper member (36), a plunger member (44), and an elongate needle member (50). The elongate needle member (50) may be constructed of metal such as stainless steel. The proximal and distal stopper members (32, 36) together with the injection system body (34) defines a proximal drug chamber (40). The distal stopper member (36) together with the injection system body (34) defines a distal drug chamber (42). The injection system body (34) includes a needle hub assembly (606) at a distal end thereof. The needle hub assembly (606) includes a needle hub and a needle member (50). The needle member (50) includes a sharp needle distal end/tip (48), a needle joining member (51) and a needle proximal end (53), which are mounted in position ready for injection after removal of a needle cover member (63).

As shown in FIG. 8A, the distal stopper member (36) includes a ball valve (2100). The ball valve (2100) includes a ball (2110), a ball trap (2120), and a longitudinal space (2130). The ball (2110) has lodged and dislodged states corresponding to respective closed and open states of the ball valve (2100). In the lodged state, the ball (2110) is disposed/held in a proximal end of the longitudinal space (2130). In the lodged state, the ball (2110) occludes the longitudinal space (2130), thereby preventing fluid flow therethrough and effectively closing the ball valve (2100). In the dislodged state (see FIGS. 12 and 12A), the ball (2110) is disposed in the ball trap (2120). In the dislodged state, the ball (2110) no longer occludes the longitudinal space (2130), allowing fluid flow through the distal stopper member (36) and effectively opening the ball valve (2100). With the ball 2110) in the dislodged state, the longitudinal space (2130) and the ball trap (2120) form a flow path through the distal stopper member (36).

The distal stopper member (36) may be formed from an elastic material, which may include one or more of the following materials: rubber, butyl rubber, chlorobutyl rubber, bromobutyl rubber, thermoplastic elastomer (TPE), thermoplastic urethane (TPU) silicone rubber, thermoplastic, Polytetrafluoroethylene (PTFE), thermoset plastic. The distal stopper member (36) may be coated with a lubricious polymer such as PTFE, silicone oil, and/or other lubricious coatings. The ball (2110) may be formed from a material impermeable to liquid which may include one or more of the following materials: polymers, cyclic olefin polymer (COP), cyclic olefin copolymer (COC), PTFE, elastomers, TPE, thermoplastic urethane (TPU), rubber, butyl rubber, chlorobutyl rubber, bromobutyl rubber, metal, stainless steel, titanium, ceramic, glass or a combination thereof.

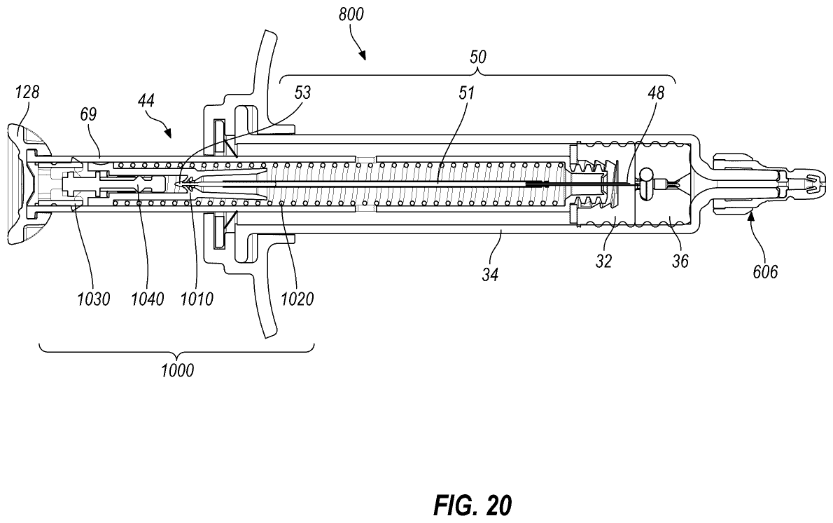

As shown in FIG. 8, the injection system (800) also includes a needle retraction system (1000) configured to withdraw/pull/retract the sharp distal end (48) of the needle member (50) to a safe position within the syringe body (34), the needle hub assembly (606), or the plunger member (44) (see e.g., FIG. 20). The needle retraction system (1000) includes a needle retention member (1010) configured to capture the needle proximal end (53), and energy-storage member/spring (1020), an energy-storage member/spring latching member (1030), and an actuating member (1040). Automatic retraction of the needle at least partially within the plunger is described in U.S. utility patent application Ser. No. 14/696,342, which was previously incorporated by reference herein.

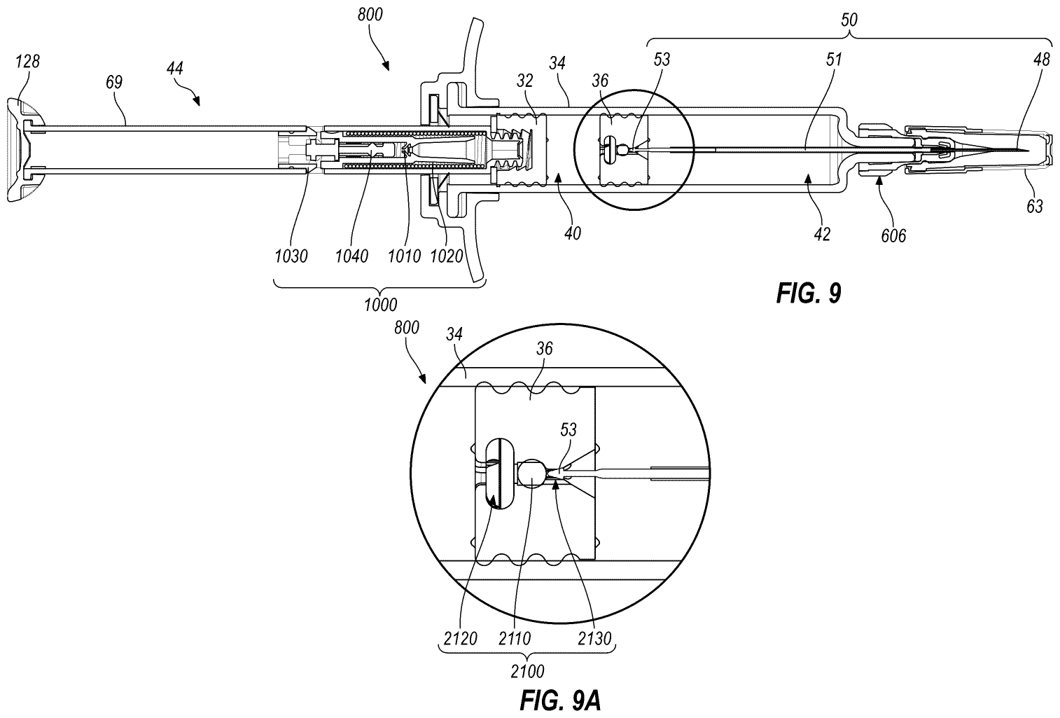

FIGS. 9 and 9A depict the safe dual chamber injection system (800) after the plunger member (44) and the proximal stopper member (32) coupled thereto have been advanced into the injection system body (34). Due to the in compressibility of the liquid in the proximal drug chamber (40), advancing the plunger member (44) and the proximal stopper member (32) distally advances the distal stopper member (36) distally into the injection system body (34). As shown in FIG. 9A, the needle proximal end (53) is partially positioned in the longitudinal space (2130) adjacent the ball (2110). The ball (2110) is still in the lodged state, and the ball valve (2100) is in the corresponding closed state.

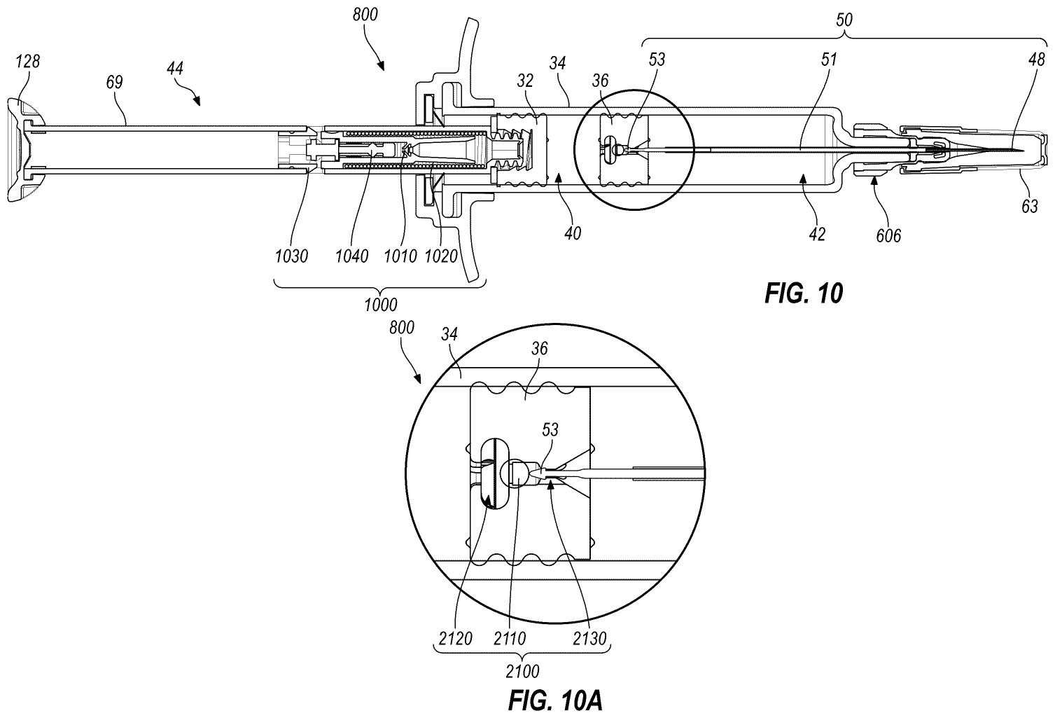

FIGS. 10 and 10A depict the safe dual chamber injection system (800) after the plunger member (44), the proximal stopper member (32) coupled thereto, and the distal stopper member (36) have been advanced further into the injection system body (34). As shown in FIG. 10A, the needle proximal end (53) is pushing the ball (2110) partially out of the longitudinal space (2130). The ball (2110) is transitioning from the lodged state to the dislodged state, and the ball valve (2100) is transitioning from the corresponding closed state to the corresponding open state.

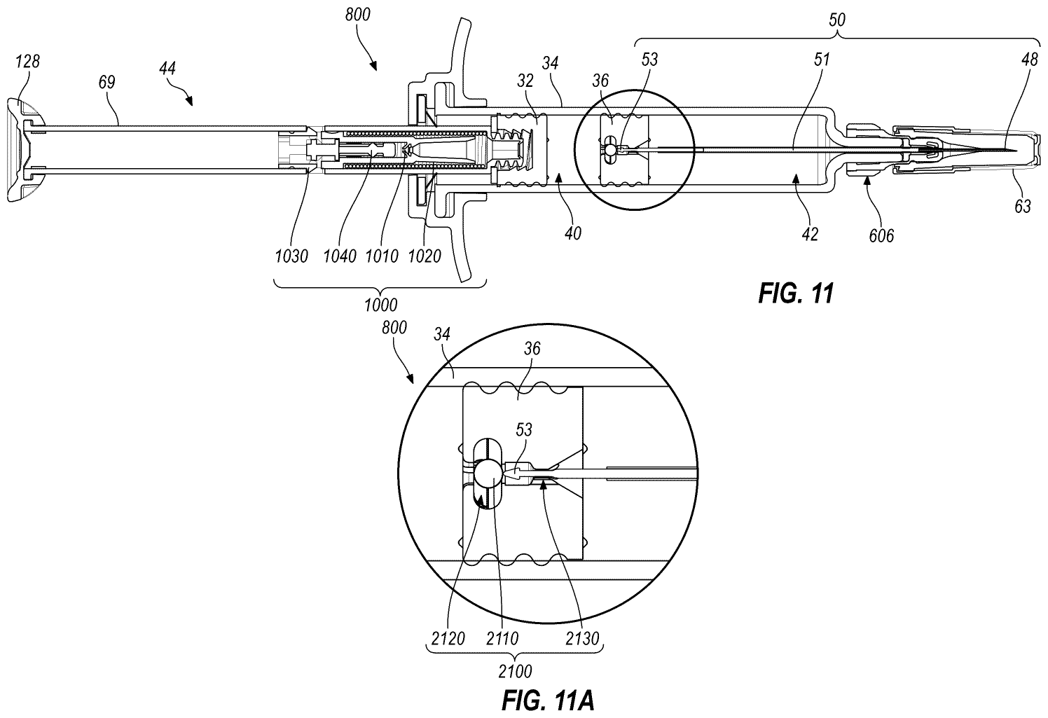

FIGS. 11 and 11A depict the safe dual chamber injection system (800) after the plunger member (44), the proximal stopper member (32) coupled thereto, and the distal stopper member (36) have been advanced still further into the injection system body (34). As shown in FIG. 11A, the needle proximal end (53) has pushed the ball (2110) out of the longitudinal space (2130). However, the ball (2110) is still in the flow path through the distal stopper member (36) and partially occludes the flow path. While the ball (2110) has transitioned from the lodged state to the dislodged state, the ball valve (2100) is continuing to transition from the corresponding closed state to the corresponding (fully) open state.

FIGS. 12 and 12A depict the safe dual chamber injection system (800) after the plunger member (44), the proximal stopper member (32) coupled thereto, and the distal stopper member (36) have been advanced yet further into the injection system body (34). As shown in FIG. 12A, the needle proximal end (53) has pushed the ball (2110) out of the longitudinal space (2130), and fluid flow through the flow path in the distal stopper member (36) has pushed the ball (2110) out of the flow path and into a side of the ball trap (2120). The ball (2110) no longer occludes the flow path. The ball (2110) has transitioned from the lodged state to the dislodged state, the ball valve (2100) has transitioned from the corresponding closed state to the corresponding open state. In this configuration, there is very low resistance to fluid flow through the ball valve (2100).

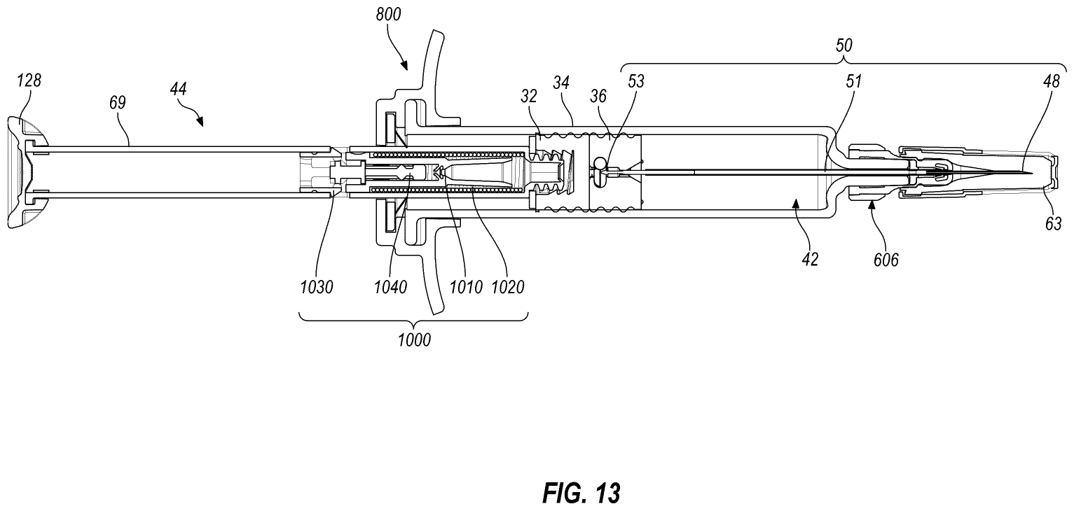

FIG. 13 depicts the safe dual chamber injection system (800) after the plunger member (44) and the proximal stopper member (32) coupled thereto have been advanced still further into the injection system body (34). Due to the opening of the ball valve (2100) the fluid in the proximal drug chamber (40, see FIG. 12) moves through the flow path in the distal stopper member (36). In FIG. 13, all of the fluid in the proximal drug chamber (40) has moved through the ball valve (2100) and the flow path in the distal stopper member (36) to the distal drug chamber (42).

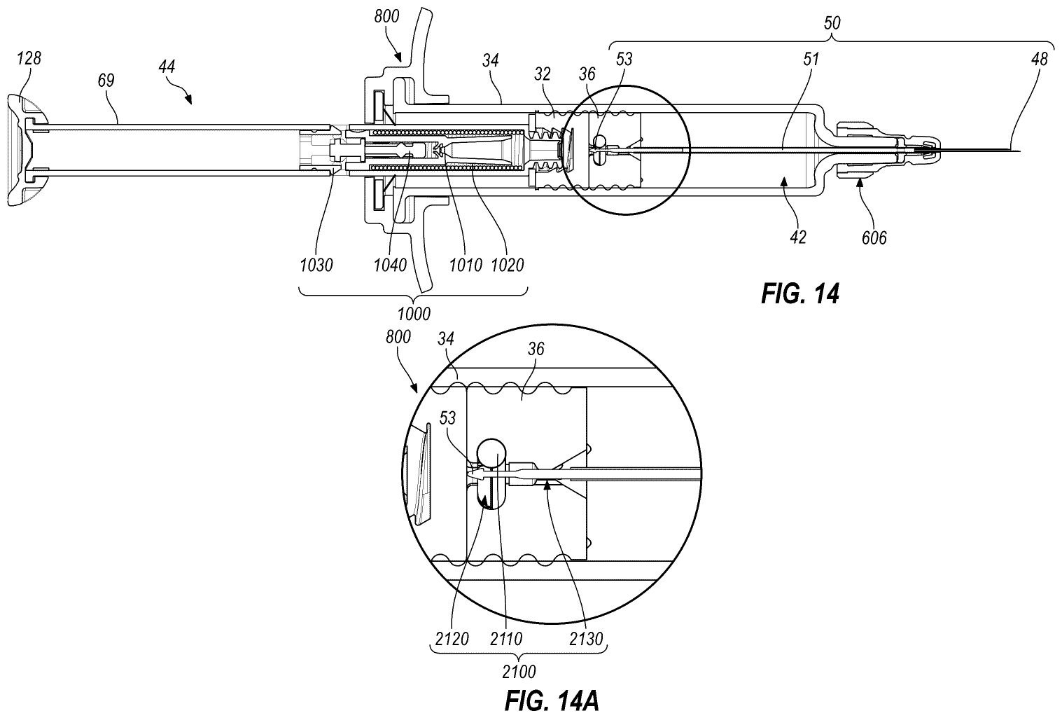

FIGS. 14 and 14A depict the safe dual chamber injection system (800) after the plunger member (44), the proximal stopper member (32) coupled thereto, and the distal stopper member (36) have been advanced yet further into the injection system body (34). As shown in FIG. 14A, the needle proximal end (53) has advanced further into the ball trap (2120), thereby retaining the ball (2110) in a side of the ball trap (2120).

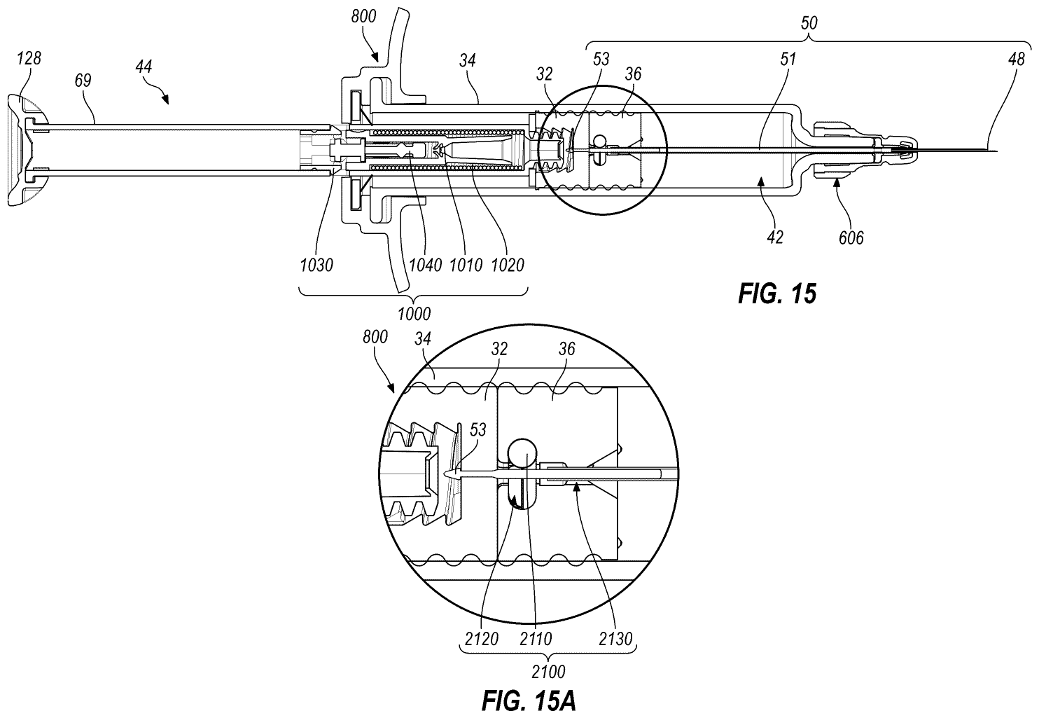

FIGS. 15 and 15A depict the safe dual chamber injection system (800) after the plunger member (44), the proximal stopper member (32) coupled thereto, and the distal stopper member (36) have been advanced still further into the injection system body (34). Advancement of the distal stopper member (36) ejects a mixed liquid from the distal drug chamber (42) out of the injection system (800) through the sharp needle distal end (48). As shown in FIG. 15A, the needle proximal end (53) has pierced the proximal stopper member (32).

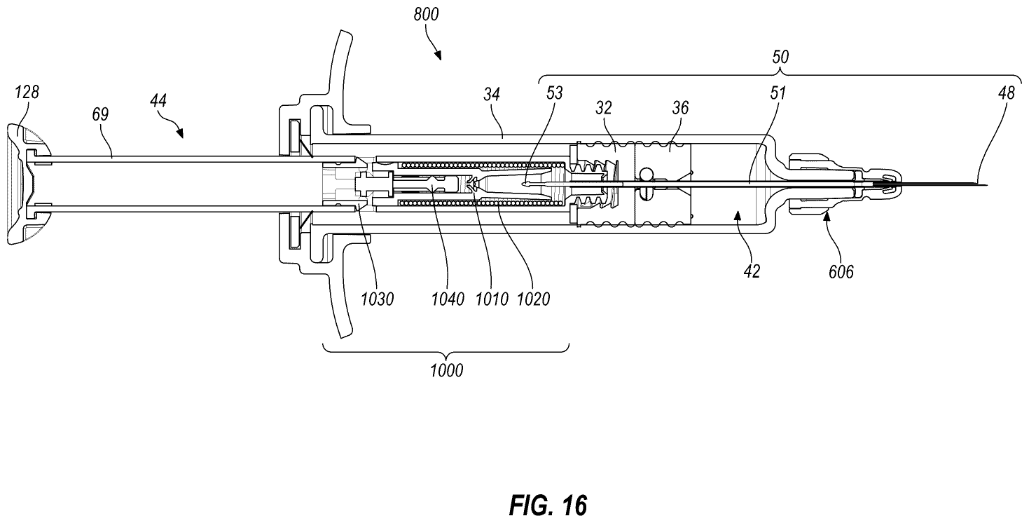

FIG. 16 depicts the safe dual chamber injection system (800) after the plunger member (44), the proximal stopper member (32) coupled thereto, and the distal stopper member (36) have been advanced yet further into the injection system body (34). Continued advancement of the distal stopper member (36) continues to eject a mixed liquid from the distal drug chamber (42) out of the injection system (800) through the sharp needle distal end (48). The needle proximal end (53) continues to advance into the plunger member (44) and the needle retraction system (1000) contained within the plunger housing member (69).

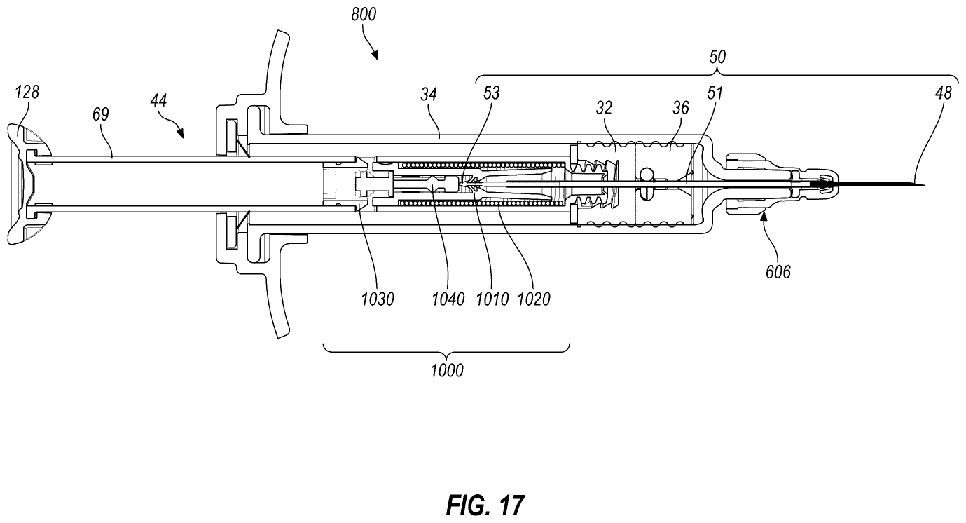

FIG. 17 depicts the safe dual chamber injection system (800) after the plunger member (44), the proximal stopper member (32) coupled thereto, and the distal stopper member (36) have been advanced still further into the injection system body (34). Continued advancement of the distal stopper member (36) continues to eject substantially all of the mixed liquid from the distal drug chamber (42) out of the injection system (800) through the sharp needle distal end (48). The needle proximal end (53) continues to advance into the plunger member (44) and the needle retraction system (1000) contained within the plunger housing member (69). In the state depicted in FIG. 17, the needle proximal end (53) has been captured by the needle retention member (1010) and is in contact with the actuating member (1040).

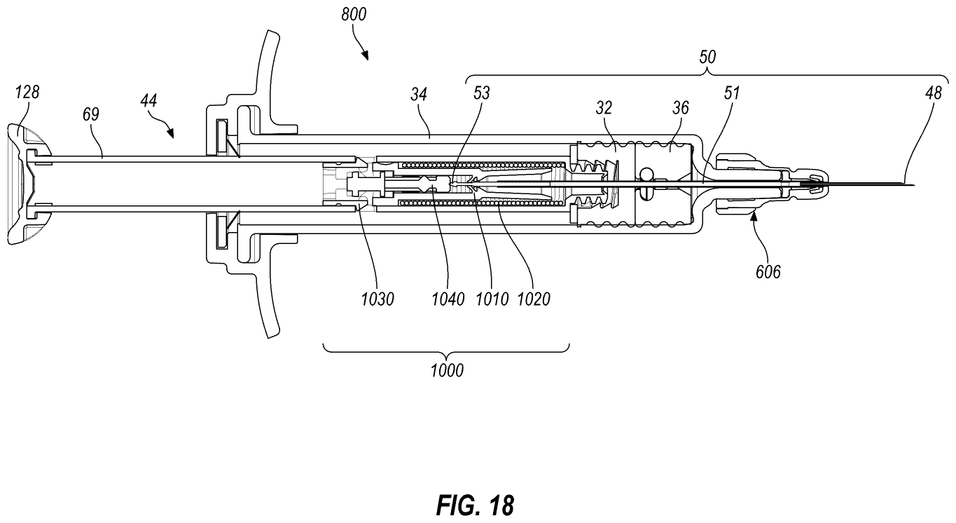

FIG. 18 depicts the safe dual chamber injection system (800) after the plunger member (44), the proximal stopper member (32) coupled thereto, and the distal stopper member (36) have been advanced yet further into the injection system body (34). The needle proximal end (53) continues to advance into the plunger member (44) and the needle retraction system (1000) contained within the plunger housing member (69). In the state depicted in FIG. 18, the needle proximal end (53) has been captured by the needle retention member (1010) and has moved the actuating member (1040) to release the energy-storage member/spring latching member (1030) and the energy-storage member/spring (1020) operatively coupled thereto.

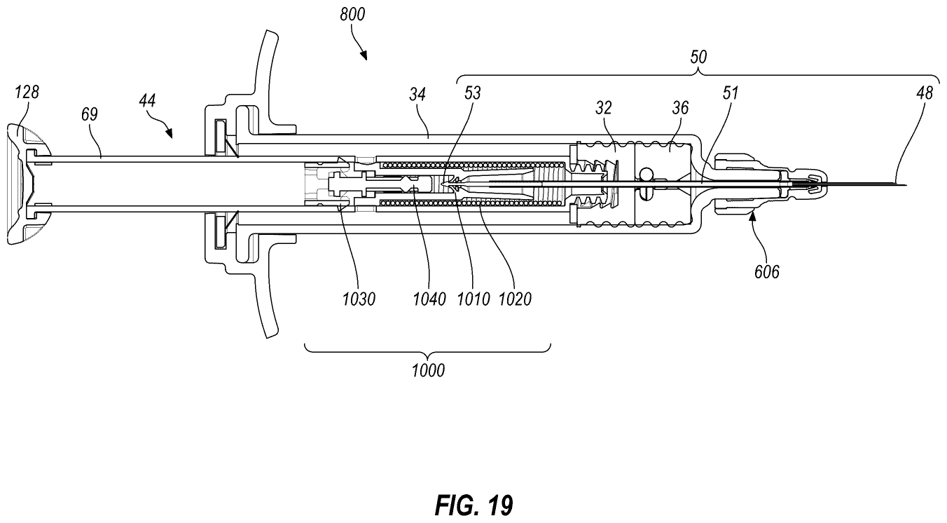

FIG. 19 depicts the safe dual chamber injection system (800) after the released the energy-storage member/spring latching member (1030) allows the energy-storage member/spring (1020) operatively coupled thereto to begin retracting the needle member (50) proximally via the interaction between the needle proximal member (53) captured by the needle retention member (1010).

FIG. 20 depicts the safe dual chamber injection system (800) after the released energy-storage member/spring (1020) has completely retracted the needle member (50) proximally via the interaction between the needle proximal member (53) captured by the needle retention member (1010). In the state depicted in FIG. 20, the sharp needle distal end (48) is disposed in the injection system body (34), thereby rendering the injection system (800) safe for disposal.

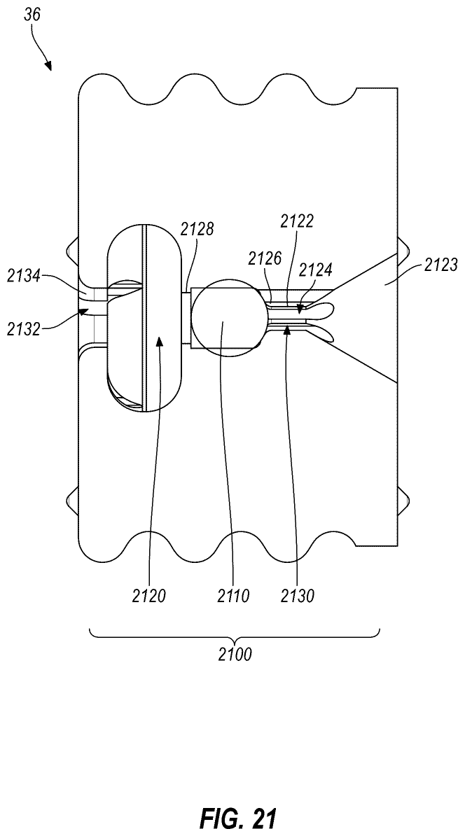

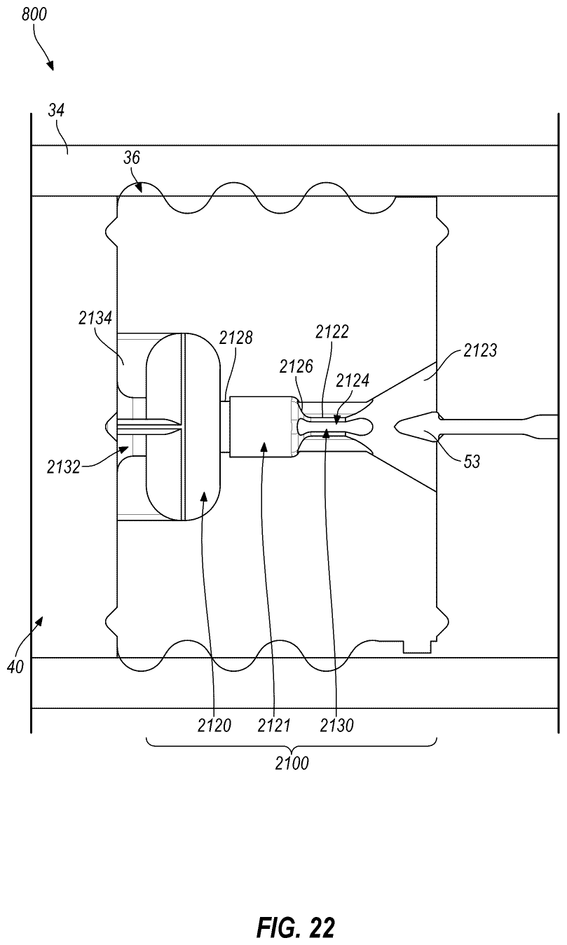

FIGS. 21 and 22 are detailed longitudinal cross-sectional views illustrating various aspects of a distal stopper member (36) with a ball (2110) disposed therein for use with a safe dual chamber injection system (800, see e.g., FIG. 8) according to some embodiments. In FIG. 22, the ball (2110) is omitted for clarity. The distal stopper member (36) includes a ball valve (2100), which in turn includes a ball (2110), a longitudinal space (2130), and a ball trap (2120). In some embodiment, the ball (2110) is a conventional 2 mm metal ball available through medical device supply channels.

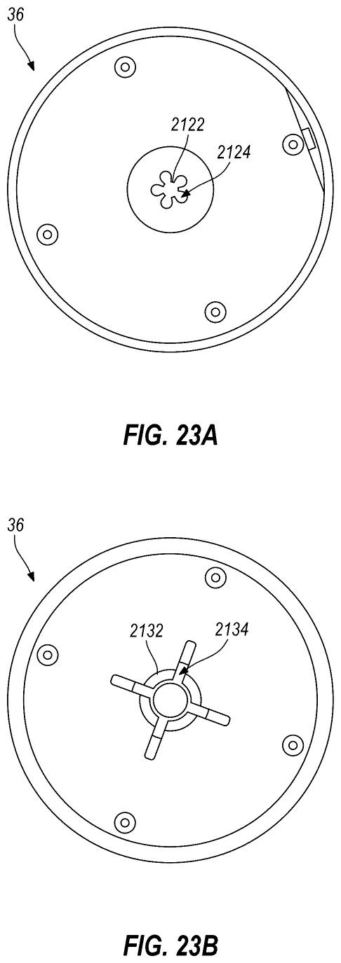

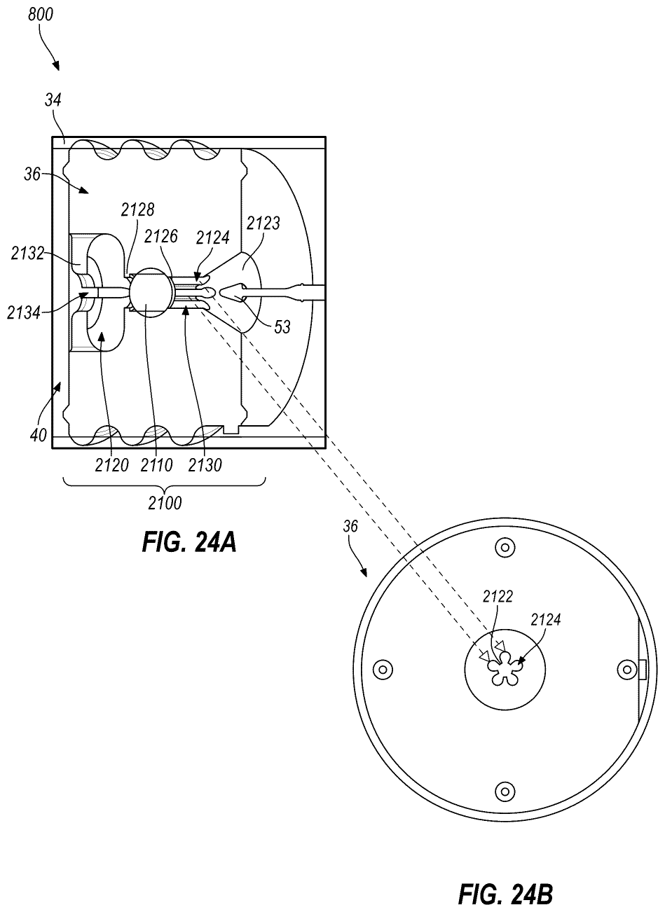

The longitudinal space (2130) includes a plurality of inwardly extending centering ribs (2122) and a corresponding plurality of flow channels (2124). The plurality of centering ribs (2122) are configured to align the needle proximal end (53) in the longitudinal space (2130) to aim the needle proximal end (53) at a center of the ball (2110) to facilitate dislodging the ball (2110) from the proximal end (2121) of the longitudinal space (2130) without the needle proximal end (53) slipping off center. Each of the plurality of flow channels (2124) is defined between a pair of adjacent centering ribs (2122) as shown in FIGS. 23A and 24B. The plurality of flow channels (2124) facilitate fluid flow around an outer diameter of the needle member (50) when the ball valve (2100) is in an open state. The plurality of centering ribs (2122) also define a corresponding plurality of rib recesses (2126) at respective proximal ends thereof. The longitudinal space (2130) also includes a plurality of inwardly extending exit ribs (2128) at a proximal end (2121) of the longitudinal space (2130). The rib recesses (2126) and the exit ribs (2128) cooperate to hold the ball (2110) in the proximal end (2121) of the longitudinal space (2130) when the ball is in the lodged state and the ball valve (2100) is in the closed state.

The distal stopper member (36) also defines a distally facing funnel (2123) configured to guide a needle proximal end (53) into the longitudinal space (2130) in which the needle proximal end (53) is aligned by the plurality of centering ribs (2122) during use of the injection system (800). The distally facing funnel (2123) is also configured to guide the needle proximal end (53) to the radial center of the longitudinal space (2130) during assembly of the injection system (800). During assembly, the needle proximal end (53) may fall into one of the plurality of flow channels (2124). The shape of each flow channel (2124) is configured to push the needle proximal end (53) radially out of the flow channel (2124) and into the radial center of the longitudinal space (2130) defined by the centering ribs (2122). While the distal stopper member (36) depicted in some FIGS. 21 to 24B include a plurality of centering ribs (2122), in other embodiments the distal stopper does not include centering ribs.

The ball trap (2120) is defined by a plurality of ball trap flaps (2132) at a proximal end thereof. The plurality of ball trap flaps (2132) also define a corresponding plurality of ball trap slots (2134), with each ball trap slot (2134) disposed between a pair of ball trap flaps (2132) as shown in FIG. 23B. The plurality of ball trap flaps (2132) are configured to hold the ball (2110) in the ball trap (2120) when the ball (2110) is in the dislodged state, and the ball valve (2100) is in the corresponding open state. The ball trap flaps (2132) and ball trap slots (2134) are also configured to facilitate removal of a metal core used in the molding process to form the ball trap (2120) after completion of the molding process.

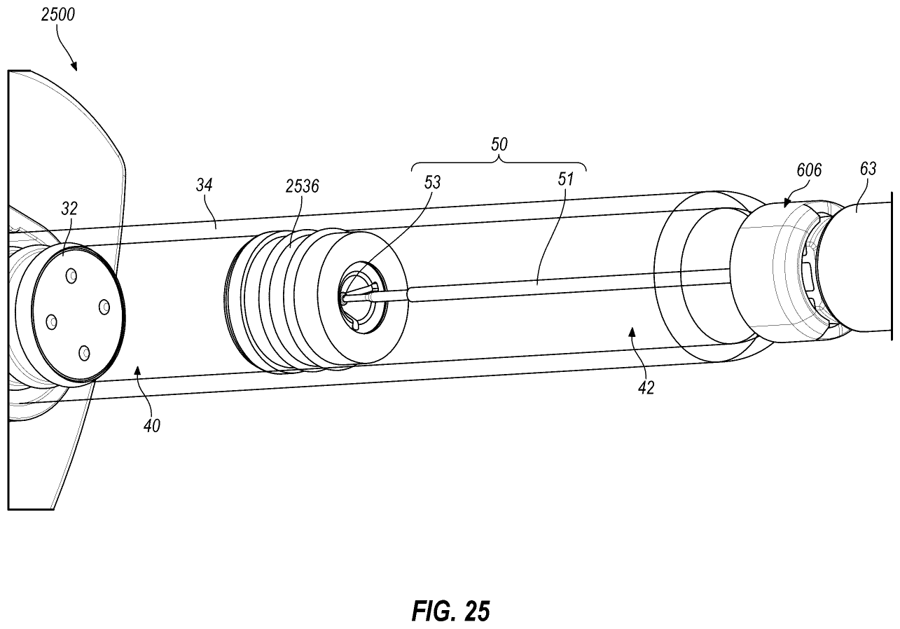

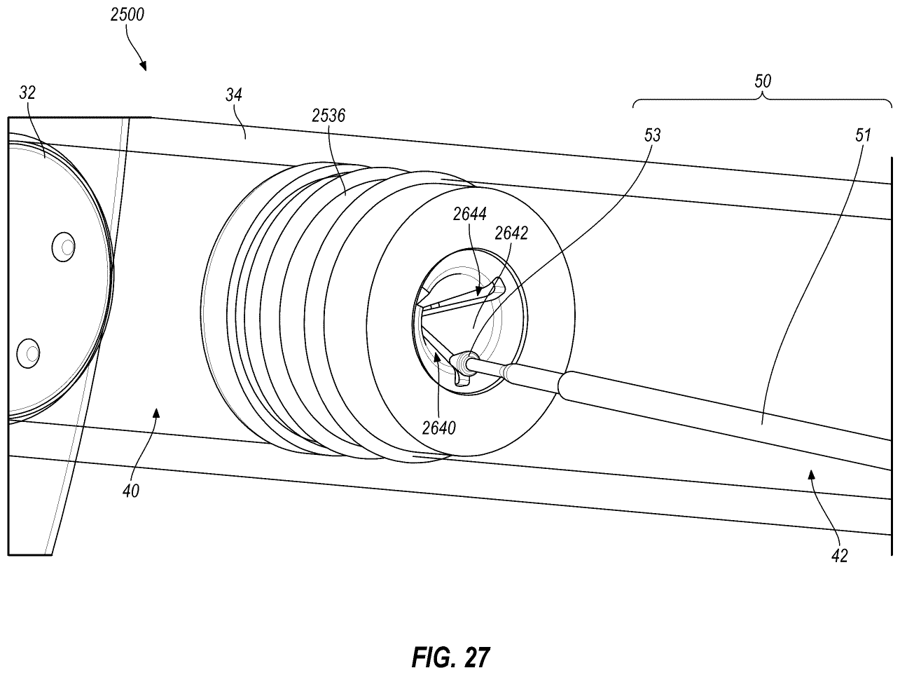

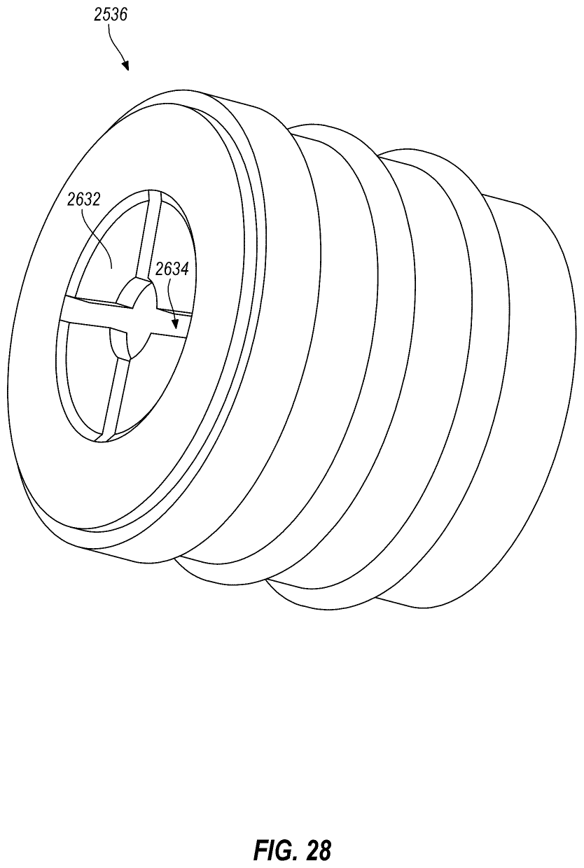

FIGS. 25 to 32 depict various aspects of syringe based dual chamber safe injection systems including a ball valve according to some embodiments. FIGS. 25 to 27 are distal perspective, detailed distal perspective cross-sectional, and detail distal perspective views illustrating various aspects of a syringe based dual chamber safe injection system (2500) according to some embodiments. The injection system (2500) depicted in FIGS. 25 to 27 is almost identical to the injection system (800) depicted in FIGS. 8 to 20.

Similar to injection system (800), injection system (2500) includes a conventional off-the-shelf pre-filled syringe body (34) with a conventional proximal stopper member (32) and a distal stopper member (2536) disposed therein. The proximal and distal stopper members (32, 2536) together with the syringe body (34) define proximal and distal drug chambers (40, 42). The proximal and distal stopper members (32, 2536) define the proximal and distal ends of the proximal drug chamber (40). The distal stopper member (2536) defines a proximal end of the distal drug chamber (42). A needle hub assembly (606) is disposed at the distal end of the distal drug chamber (42) with a needle cover member (63) installed for storage. The dual chamber safe injection system (2500) controls transfer of a first drug component from the proximal drug chamber (40) to the distal drug chamber (42) and exit of a combined/mixed drug from the distal drug chamber (42) distally subject to sequential insertion of a plunger member relative to the syringe body (34) to various degrees by a user. The plunger member includes the proximal stopper member (32), a plunger housing member (69) and a plunger manipulation interface (128) (see e.g., FIG. 7A). The only difference between injection systems (2500, 800) is the ball valve (2600) in the injection system (2500) depicted in FIGS. 25 to 27.

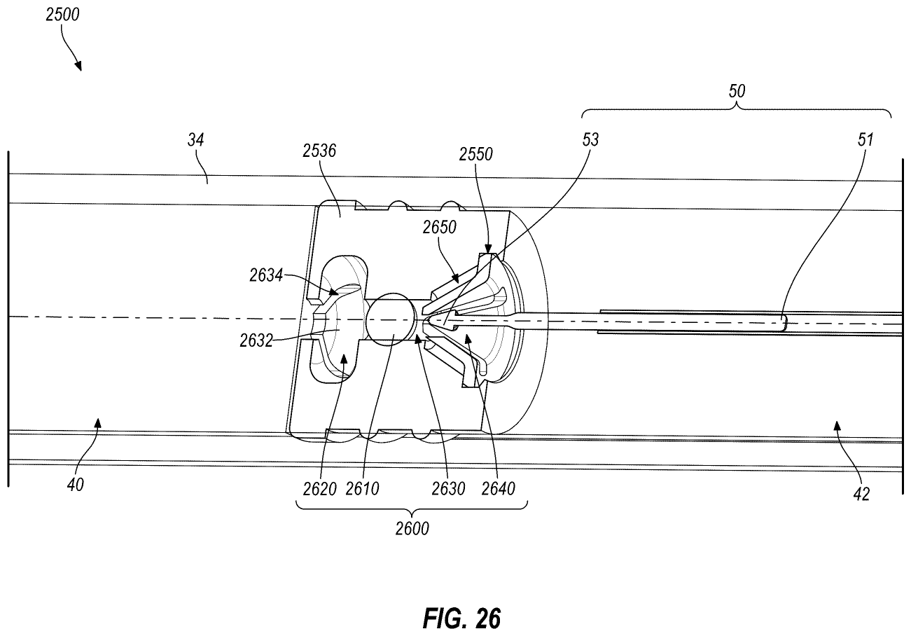

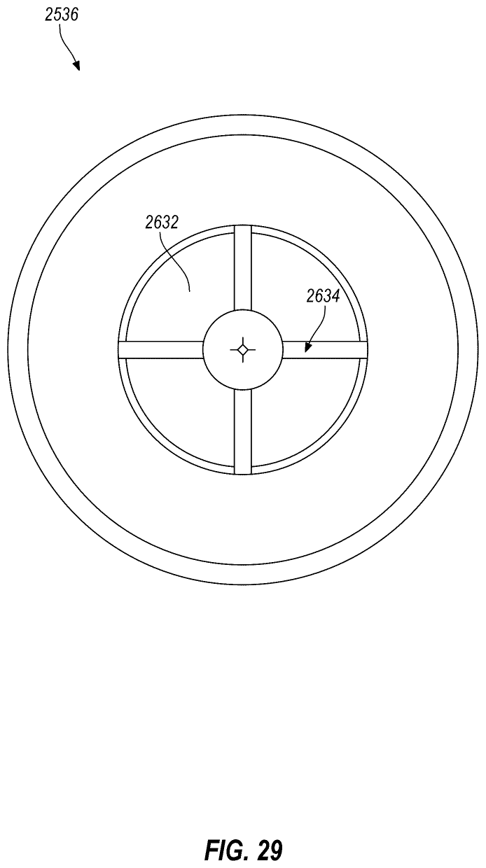

As shown in FIG. 26, the distal stopper member (2536) in the injection system (2500) includes a ball (2610), a ball trap (2620), a longitudinal space (2630), and a funnel insert (2640). The ball (2610) and the ball trap (2620) are similar to the corresponding ball (2110) and ball trap (2120) in the ball valve (2100) depicted in FIGS. 21 to 24B. For instance, the ball trap (2620) includes ball trap flaps (2622) separated by ball trap slots (2624) as shown in FIGS. 28 and 29.

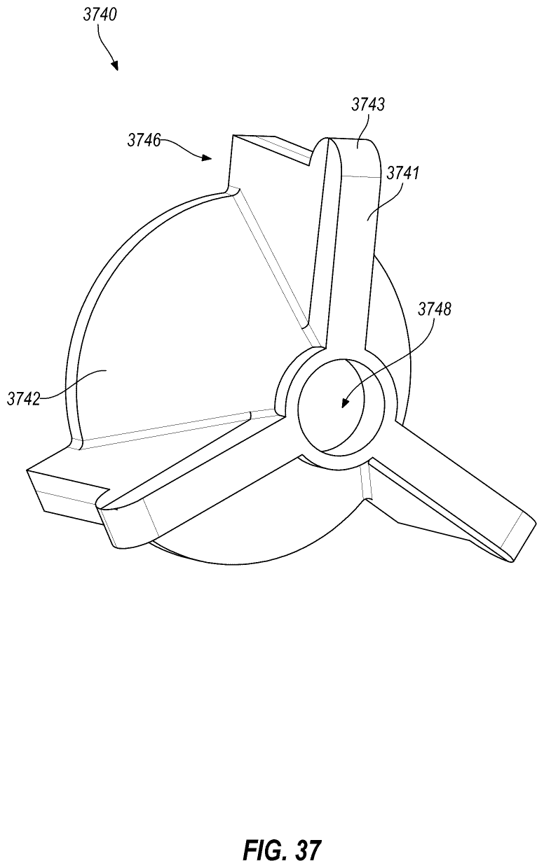

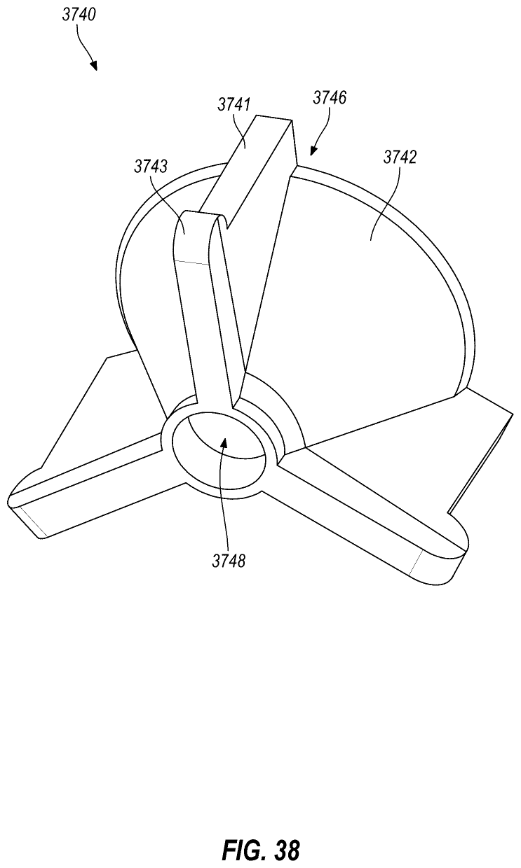

FIG. 26 depicts another centering/alignment embodiment including a funnel insert (2640) configured to guide a needle proximal end (53) into the longitudinal space (2630) and align the needle proximal end (53) therein during use of the injection system (800). The funnel insert (2640) grips the needle proximal end (53) tightly to accurately guide/center/align it in the longitudinal space (2630). Unfortunately, this close grip between the funnel insert (2640) and the needle proximal end (53) partially blocks liquid flow during liquid transfer. To address this problem, a large space (2650) is provided between the funnel insert (2640) and the elastic/rubber distal stopper member (2536) to allow the liquid to flow around this partial blockage. While the funnel insert (2640) depicted in FIGS. 25-27 grips the needle proximal end (53) tightly, in other embodiments the funnel insert defines a central opening having an inner diameter larger than an outer diameter of the needle proximal end (53). In such other embodiments, the funnel insert does not grip the needle proximal end tightly. In other embodiments, the funnel insert is configured to further increase liquid flow (see e.g., FIGS. 37-40).

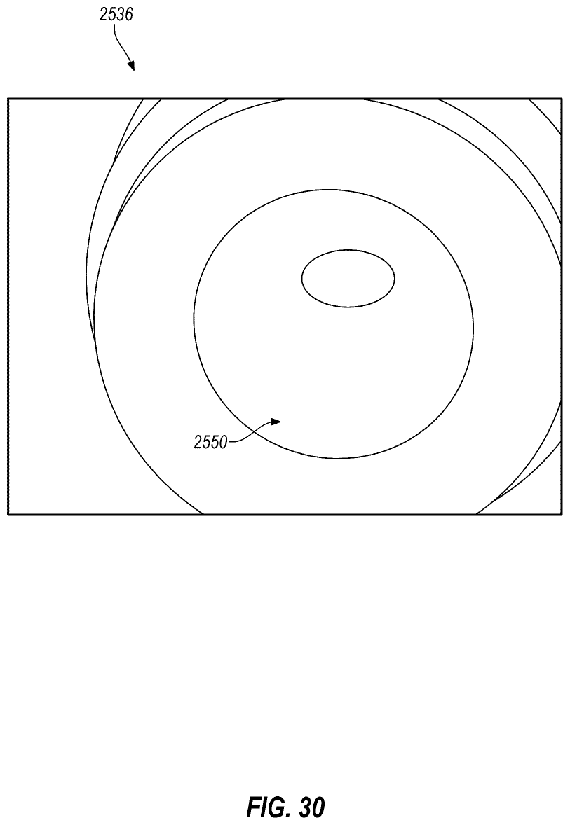

The longitudinal space (2630) in ball valve (2600) is shorter than the longitudinal space (2130) in ball valve (2100). Further, ball valve (2600) includes a funnel insert (2640). The body of the distal stopper member (2536) may be made from an elastic material and the funnel insert (2640) may be made from a rigid material (e.g., plastic) to facilitate placement of the funnel insert (2640) into an annular channel (2550) in the distal stopper member (2536) as shown in FIGS. 26 and 30. In other embodiments, the funnel insert is configured to be placed in the distal stopper member using other mechanisms (see e.g., FIGS. 37-40). The funnel insert (2640) includes a plurality of funnel flaps (2642) defining and separated by a respective plurality of funnel channels (2644). The pluralities of funnel flaps (2642) and funnel channels (2644) are configured to guide a needle proximal end (53) into the longitudinal space (2630) to dislodge the ball (2610) in a similar manner as shown for the ball valve (2100) in FIGS. 11 to 12A. Dislodging the ball (2610) moves the ball from a lodged state to a dislodged state and the ball valve (2600) from a closed state to an open state. The funnel channels (2644) in the funnel insert (2640) of ball valve (2600) allow the funnel flaps (2642) to deform around a needle proximal end (53) to center and align the needle proximal end (53). The funnel channels (2644) also provide a flow path for the fluid to flow out of the space (2650) between the distal stopper member (2536) and the funnel insert (2640). Embodiments including a funnel insert (2640) may be used for delivering viscous liquids, where a larger flow channel is preferred, and a larger ball and longitudinal space may be used. In other embodiments, the funnel insert is configured to further increase liquid flow (see e.g., FIGS. 37-40).

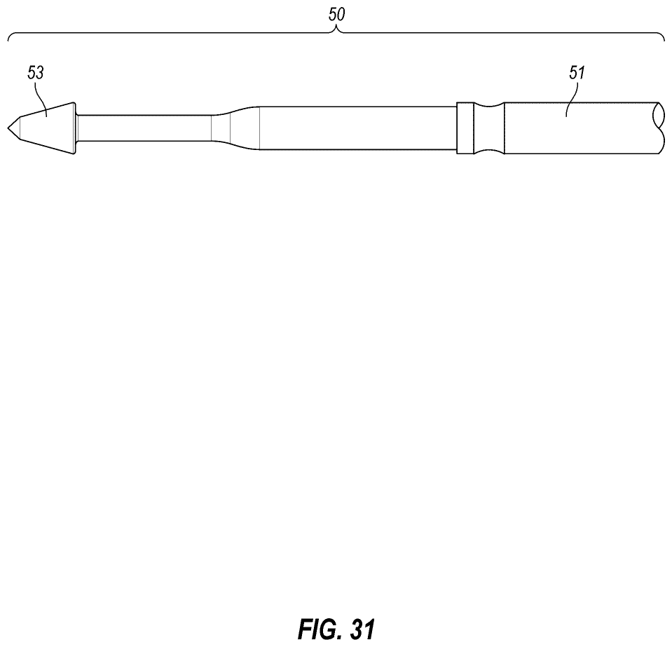

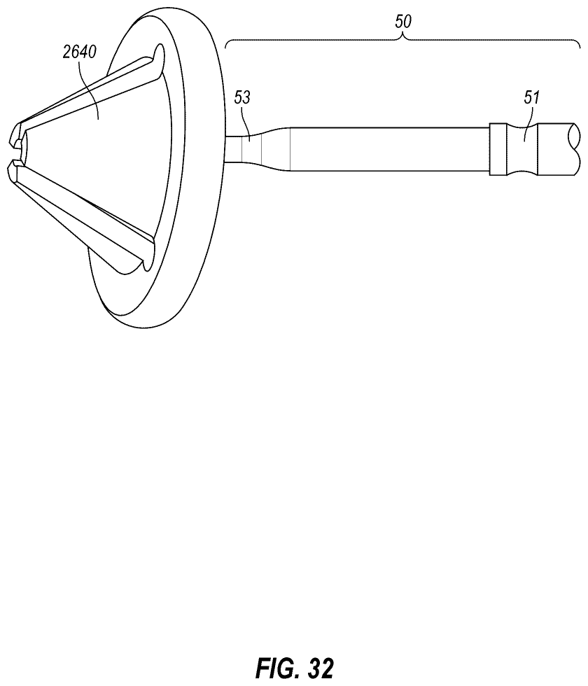

FIG. 31 is a side view illustrating various aspects of a needle (50) for use with safe syringe based dual chamber injection systems (700, 800, 2500) according to some embodiments. FIG. 32 is a side view illustrating various aspects of a needle (50) and a funnel insert (2640) for use with safe syringe based dual chamber injection systems (2500) according to some embodiments.

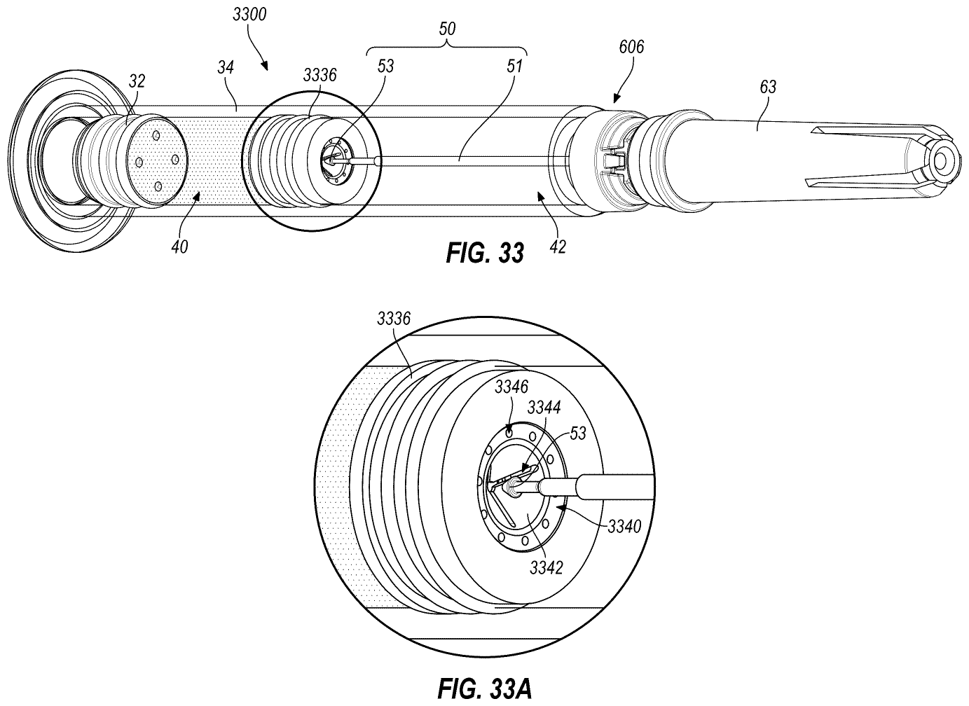

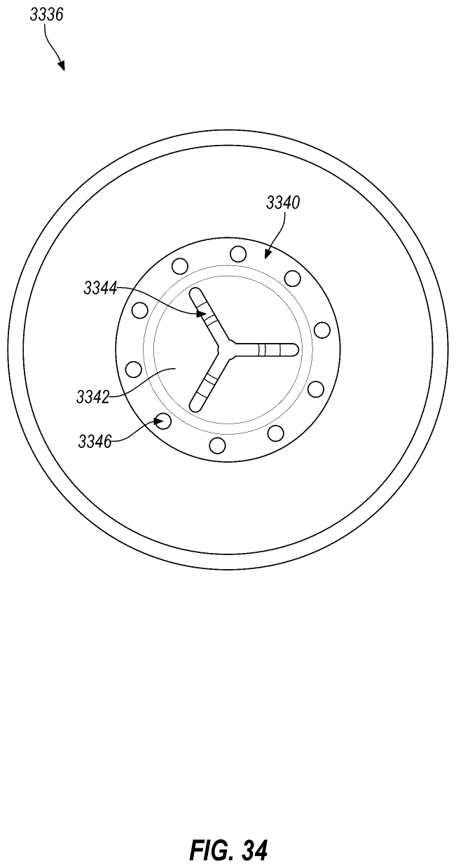

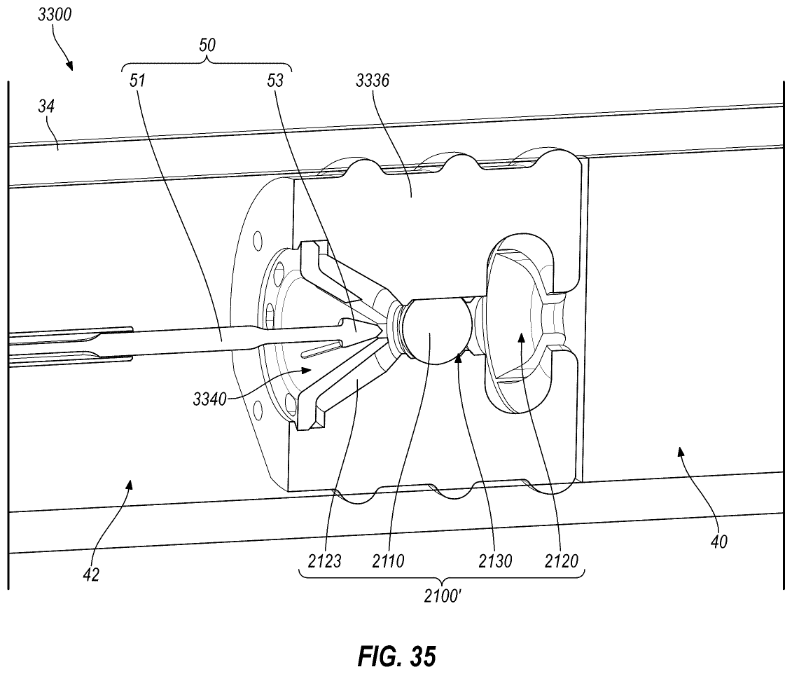

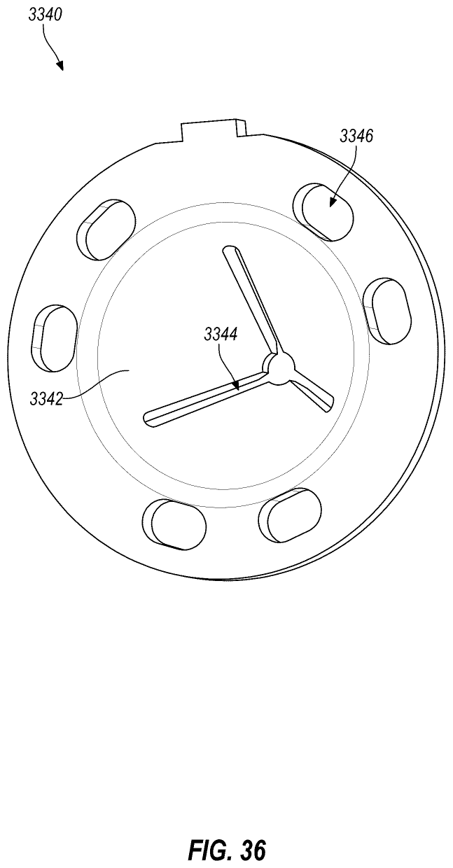

FIGS. 33, 33A, 34, 35, and 36 depict various aspects of a syringe based dual chamber safe injection system (3300) including a ball valve (2100′) according to some embodiments. FIGS. 33 and 33A are distal and detailed distal perspective views illustrating various aspects of the safe syringe based dual chamber injection system (3300) and a distal stopper member (3336) for use with same according to some embodiments. FIG. 34 is a distal axial view illustrating various aspects of the distal stopper member (3336) for use with the safe syringe based dual chamber injection system (3300) according to some embodiments. FIG. 35 is a detailed perspective cross-sectional view illustrating various aspects of the safe syringe based dual chamber injection system (3300) and distal stopper member (3336) for use with same according to some embodiments. FIG. 36 is a distal perspective view illustrating various aspects of a funnel insert (3340) for use with the distal stopper member (3336) in the safe syringe based dual chamber injection system (3300) according to some embodiments. The injection system (3300) depicted in FIGS. 33-36 is almost identical to the injection system (2500) depicted in FIGS. 25 to 27 and the injection system (800) depicted in FIGS. 8 to 20.

Similar to injection systems (800, 2500), injection system (3300) includes a conventional off-the-shelf pre-filled syringe body (34) with a conventional proximal stopper member (32) and a distal stopper member (3336) disposed therein. The proximal and distal stopper members (32, 3336) together with the syringe body (34) define proximal and distal drug chambers (40, 42). The proximal and distal stopper members (32, 3336) define the proximal and distal ends of the proximal drug chamber (40). The distal stopper member (3336) defines a proximal end of the distal drug chamber (42). A needle hub assembly (606) is disposed at the distal end of the distal drug chamber (42) with a needle cover member (63) installed for storage. The dual chamber safe injection system (3300) controls transfer of a first drug component from the proximal drug chamber (40) to the distal drug chamber (42) and exit of a combined/mixed drug from the distal drug chamber (42) distally subject to sequential insertion of a plunger member relative to the syringe body (34) to various degrees by a user. The only difference between injection systems (3300, 2500) is the funnel insert (3340) in the injection system (3300) depicted in FIGS. 33 to 36 (vs. the funnel insert (2640) in injection system 2500).

Like the funnel insert (2640), the funnel insert (3340) includes a plurality of funnel flaps (3342) defining and separated by a respective plurality of funnel channels (3344). The pluralities of funnel flaps (3342) and funnel channels (3344) are configured to guide a needle proximal end (53) into the longitudinal space (2130) to dislodge the ball in a similar manner as shown for the ball valve (2100) in FIGS. 11 to 12A. The funnel channels (3344) in the funnel insert (3340) of ball valve (2100′) allow the funnel flaps (3342) to deform around a needle proximal end (53) to center and align the needle proximal end (53). The funnel channels (3344) also provide a flow path for the fluid to flow out of the space between the distal stopper member (3336) and the funnel insert (3340).

The difference between the funnel inserts (3340, 2640) is that the funnel insert (3340) includes a plurality of flow channels (3346) to provide an additional flow path for fluid to flow from the proximal drug chamber (40) to the distal drug chamber (42), thereby increasing fluid flow. The fluid flows from the proximal drug chamber (40) out of the space between the distal stopper member (3336) and the funnel insert (3340) and distally through the pluralities of funnel channels (3344) and flow channels (3346).

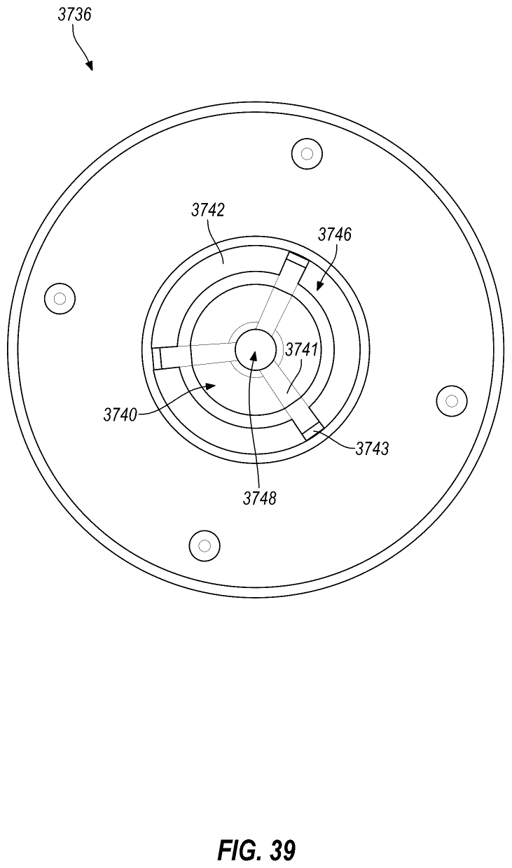

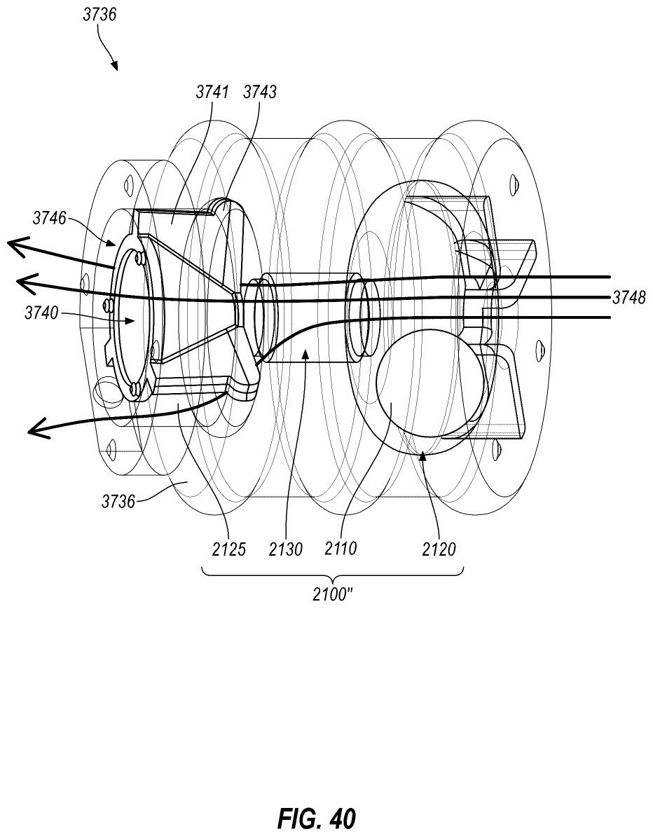

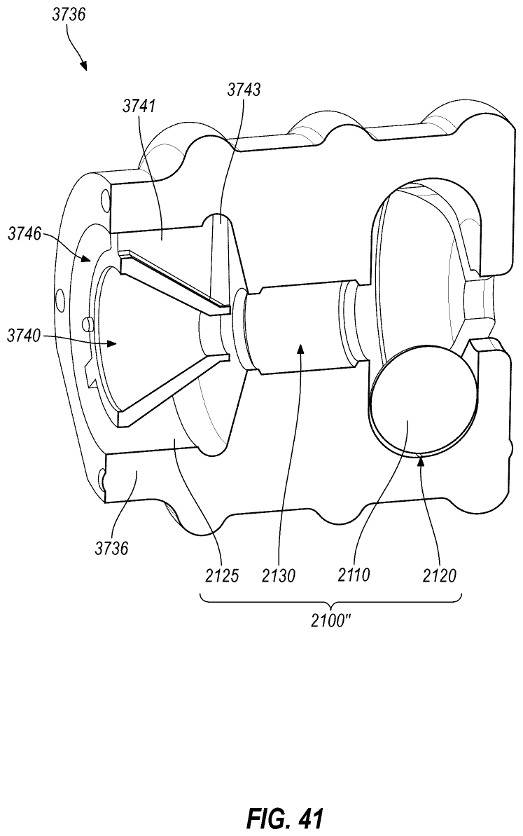

FIGS. 37, 38, 39, 40, and 41 depict various aspects of a stopper member (3736, see FIGS. 39, 40, and 41) in a syringe based dual chamber safe injection system including a ball valve (2100″) and a distally facing funnel insert (3740, see FIGS. 37 and 38) for use with same according to some embodiments. FIGS. 37 and 38 are proximal perspective views a funnel insert (3740) for use with a stopper member (3736, see FIGS. 39, 40, and 41) in safe syringe based dual chamber injection systems according to some embodiments. FIG. 39 is a distal axial view illustrating various aspects of a stopper member (3736) for use with safe syringe based dual chamber injection systems according to some embodiments. FIGS. 40 and 41 are detailed perspective and detailed perspective cross-sectional views illustrating various aspects of a stopper member (3736) having ball valves (2100″) for use in safe syringe based dual chamber injection systems according to some embodiments. The funnel insert (3740) and the stopper member (3736) may be used with various safe syringe based dual chamber injection systems (e.g., 3300; see FIGS. 33 and 35).

Injection systems (e.g., 3300) with which the funnel insert (3740) and the stopper member (3736) may be used, include a conventional off-the-shelf pre-filled syringe body (34) with a conventional proximal stopper member (32) and a distal stopper member (3736) disposed therein (see FIGS. 33 and 35). The proximal and distal stopper members (32, 3736) together with the syringe body (34) define proximal and distal drug chambers (40, 42). The proximal and distal stopper members (32, 3736) define the proximal and distal ends of the proximal drug chamber (40). The distal stopper member (3736) defines a proximal end of the distal drug chamber (42). A needle hub assembly (606) is disposed at the distal end of the distal drug chamber (42) with a needle cover member (63) installed for storage. The dual chamber safe injection system (3300) controls transfer of a first drug component from the proximal drug chamber (40) to the distal drug chamber (42) and exit of a combined/mixed drug from the distal drug chamber (42) distally subject to sequential insertion of a plunger member relative to the syringe body (34) to various degrees by a user.

The funnel insert (3740) and the stopper member (3736) depicted in FIGS. 37-41 are similar to the funnel insert (3340) and the stopper member (3336) in FIGS. 33 to 36. A difference between funnel inserts (3340 and 3740) is that funnel insert (3740) defines a central opening (3748) sized and shaped to allow a needle proximal end feature to pass therethrough, whereas the central opening in the funnel insert (3340) is sized and shaped to prevent a needle proximal end feature (53, see e.g., FIG. 35) from passing therethrough until the funnel flaps (3342) open. The funnel insert (3740) includes a continuous cone (3742), which does not need to open to allow the needle proximal end feature (53) to pass through the central opening (3748). Although the larger central opening (3748) affects alignment of the needle proximal end feature (53), the central opening (3748) is sized and shaped to allow the needle proximal end feature (53) to pass therethrough while directing the needle proximal end feature (53) toward the ball (2110, see e.g., FIG. 35) to dislodge the ball (2110) in a similar manner as shown for the ball valve (2100) in FIGS. 11 to 12A.

The funnel insert (3740) also has a plurality (e.g., three) of ribs (3741) configured to support the funnel insert (3740) in the distal stopper member (3736, see FIGS. 40 and 41). The funnel insert (3740) and the distal stopper member (3736) together define a plurality of arcuate slots (3746) adjacent an outer circumference of the funnel insert (3740) configured to provide low liquid flow resistance through the distal stopper member (3736). The funnel insert (3740) may be formed from a plastic material while the distal stoper member (3736) may be formed form an elastic material (e.g., rubber) such that the plurality of ribs (3741) can elastically deform the distal stopper member to form a cylindrical space (2125) behind the plurality of arcuate slots (3746) to facilitate liquid flow (3748, see FIG. 40). Forming the funnel insert (3740) from a plastic material also minimizes cracking.

Plastic materials from which the funnel insert (3740) may be formed include polymer material such as cyclic olefin polymer (COP) or cyclic olefin copolymer (COC). In other embodiments, the funnel insert (3740) may be formed from other polymer materials, metal, ceramic, or glass, materials, or a combination thereof. Elastic materials from which the distal stopper member (3736) may be formed include one or more of the following materials: rubber, butyl rubber, chlorobutyl rubber, bromobutyl rubber, thermoplastic elastomer (TPE), thermoplastic urethane (TPU) silicone rubber, thermoplastic, Polytetrafluoroethylene (PTFE), thermoset plastic.

As shown in FIGS. 40 and 41, the distal stopper member (3736) includes a ball valve (2100″). The ball valve (2100″) includes a ball (2110), a ball trap (2120), a longitudinal space (2130), and the cylindrical space (2125). The ball (2110) has lodged and dislodged states corresponding to respective closed and open states of the ball valve (2100″). In the lodged state, the ball (2110) is disposed/held in a proximal end of the longitudinal space (2130). In the lodged state, the ball (2110) occludes the longitudinal space (2130), thereby preventing fluid flow therethrough and effectively closing the ball valve (2100″). In the dislodged state (see FIGS. 40 and 41), the ball (2110) is disposed in the ball trap (2120). In the dislodged state, the ball (2110) no longer occludes the longitudinal space (2130), allowing fluid flow through the distal stopper member (3736) and effectively opening the ball valve (2100″). With the ball 2110) in the dislodged state, the longitudinal space (2130) and the ball trap (2120) form a flow path (3748) through the distal stopper member (36).

While the embodiments described above include dual chamber safety injection systems, the scope of the claims also include other multiple chamber safety injection systems. For multiple chamber safety injection systems with more than two chambers, more than two stopper members are inserted into an injection system body (e.g., syringe body, cartridge body, etc.) to define a corresponding number of chambers.

While the prefilled dual chamber safety injection systems depicted and described herein include staked needles, the various configurations/embodiments described herein (e.g., serial injection, detent dual chamber, threaded plunger member, and shielded and vented needle cover) can be used with cartridges, auto injectors, and injection systems with Luer connectors, transfer pipes, and no needles such as those described in U.S. Utility patents application Ser. No. 15/801,281 and Ser. No. 15/801,259, which were previously incorporated by reference herein.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. An injection system, comprising:
an injection system body defining a proximal opening at a proximal end thereof and a distal needle interface at a distal end thereof;
a proximal stopper member and a distal stopper member disposed in the injection system body, wherein the proximal stopper member, the distal stopper member, and the injection system body form
a proximal drug chamber between the proximal stopper member and the distal stopper member, and
a distal drug chamber between the distal stopper member and the distal end of the injection system body;
a plunger member configured to insert the proximal stopper member relative to the injection system body; and
a needle hub assembly coupled to the syringe body at the syringe body distal end, and comprising
a needle hub, and
a needle removably coupled to the needle hub and having a sharp needle distal end and a needle proximal end feature,
wherein the distal stopper member comprises a ball having a lodged state and a dislodged state,
wherein, when the ball is in the lodged state, the ball forms an openable barrier between the proximal and distal drug chambers, and
wherein the distal stopper member is configured to selectively allow flow from the proximal drug chamber to the distal drug chamber.

2. The system of claim 1, wherein the distal stopper member defines a ball trap disposed proximal of the ball when the ball is in the lodged state.

3. The system of claim 2, wherein the distal stopper member comprises a plurality of ball trap flaps separated by ball trap slots, and
wherein the ball trap flaps are configured to retain the ball in the ball trap when the ball is in the dislodged state.

4. The system of claim 1, wherein the distal stopper member defines a longitudinal space configured to hold the ball in a proximal end thereof when the ball is in the lodged state,
wherein the distal stopper member comprises a plurality of inwardly extending exit ribs at the proximal end of the longitudinal space, and
wherein the plurality of inwardly extending exit ribs is configured to hold the ball in the proximal end of the longitudinal space when the ball is in the lodged state.

5. The system of claim 4, wherein the distal stopper member defines a plurality of flow channels along an interior wall of a distal end of the longitudinal space,
wherein the distal stopper member comprises a plurality of inwardly extending centering ribs between corresponding pairs of flow channels,
wherein the plurality of inwardly extending centering ribs are configured to align the needle in the longitudinal space, and
wherein the plurality of inwardly extending centering ribs are configured to retain the ball in the proximal end of the longitudinal space when the ball is in the lodged state.

6. The system of claim 5, wherein each of the plurality of inwardly extending centering ribs defines a respective rib recess configured to retain the ball in the proximal end of the longitudinal space when the ball is in the lodged state.

7. The system of claim 5, wherein the distal stopper member defines a distally opening funnel at a distal end thereof,
wherein the distally opening funnel is configured to guide the needle proximal end feature and the needle into a distal opening of the longitudinal space.

8. The system of claim 1, wherein the plunger member comprises
a plunger member body defining a plunger interior,
a needle retention member disposed in the plunger interior and configured to couple to the needle proximal end feature,
an energy-storage member disposed in the plunger interior and configured to withdraw the needle retention member proximally in the plunger interior, and
an energy-storage member latching member disposed in the plunger interior and having
a latched state in which the energy-storage member latching member holds the energy-storage member in an energized state, and
an unlatched state in which the energy-storage member latching member does not restrain the energy-storage member and the energy-storage member transforms into a released state, and
wherein when the distal stopper member is inserted to the syringe body distal end, the energy-storage member latching member is transformed from the latched state to the unlatched state, and the energy-storage member pulls needle proximally such that the sharp needle distal end is disposed in an interior of the injection system body.

9. The system of claim 8, wherein when the distal stopper member is inserted to the syringe body distal end, the energy-storage member latching member is transformed from the latched state to the unlatched state, and the energy-storage member pulls the needle proximally via the needle retention feature coupled to the needle proximal end feature.

10. The system of claim 1, wherein the distal stopper member comprises a distally opening funnel insert at a distal end thereof,
wherein the distally opening funnel insert is configured to guide the needle proximal end feature and the needle into a distal opening of the longitudinal space.

11. The system of claim 10, wherein the distally opening funnel insert defines a central opening sized and shaped to allow the needle proximal end feature to pass therethrough,
wherein the distally opening funnel insert comprises a plurality of ribs configured to support the distally opening funnel insert in the distal stopper member, and
wherein the distally opening funnel insert and the distal stopper member define a plurality of arcuate slots adjacent an outer circumference of the distally opening funnel insert configured to provide low liquid flow resistance through the distal stopper member.

12. The system of claim 10, wherein the distally opening funnel insert is formed from a plastic material.

13. A method for injecting, comprising:
providing a prefilled injection system, the injection system comprising
an injection system body defining a proximal opening at a proximal end thereof and a distal needle interface at a distal end thereof,
a proximal stopper member and a distal stopper member disposed in the injection system body, wherein the proximal stopper member, the distal stopper member, and the injection system body form
a proximal drug chamber between the proximal stopper member and the distal stopper member, and
a distal drug chamber between the distal stopper member and the distal end of the injection system body,
a plunger member configured to insert the proximal stopper member relative to the injection system body,
a needle hub assembly coupled to the syringe body at the syringe body distal end, and comprising
a needle hub, and
a needle removably coupled to the needle hub and having a sharp needle distal end and a needle proximal end feature,
a first drug component disposed in the proximal drug chamber, and
a second drug component disposed in the distal drug chamber,
wherein the distal stopper member comprises a ball having a lodged state and a dislodged state;
moving the plunger and the proximal stopper member coupled thereto distally into the injection system body to move the distal stopper member distally into the injection system body and to dispose the needle proximal end feature in the distal stopper member adjacent the ball;
moving the plunger and the proximal stopper member coupled thereto distally further into the injection system body to dislodge the ball from the distal stopper member using the needle proximal end feature;
moving the plunger and the proximal stopper member coupled thereto distally yet further into the injection system body to transfer the first drug component from the proximal drug chamber to the distal drug chamber to form a mixed drug with the second drug component; and
moving the plunger and the proximal stopper member coupled thereto distally still further into the injection system body to eject the mixed drug from the distal drug chamber through the needle.

14. The method of claim 13, further comprising automatically retracting the needle proximally after ejecting the mixed drug from the distal drug chamber such that the sharp needle distal end is disposed in an interior of the injection system body.
